(19) United States
(12) Patent Application Publication
(10) Pub. No.: US 2010/0048714 A1
(43) Pub. Date: Feb. 25, 2010

(54) Methods and Compositions Related to Inhibition of Ceramide Synthesis

(75) Inventor: Scott A. Summers, Singapore (SG)

Correspondence Address: Ballard Spahr LLP, SUITE 1000, 999 PEACHTREE STREET, ATLANTA, GA, 30309-3915, US

(73) Assignee: University of Utah Research Foundation, Salt lake City, UT (US)

(21) Appl. No.: 12/305,772

(22) PCT Filed: Jun. 19, 2007
(86) PCT No.: PCT/US07/71576

§ 371 Date: Dec. 19, 2008

Related U.S. Application Data
(60) Provisional application No. 60/814,932, filed on Jun. 19, 2006.

(51) Int. Cl. A61K 31/16; C12N 5/00; A61K 31/05; C12Q 1/02; C40B 30/08; C07C 233/02; C07C 39/12; G01N 33/00; A61P 3/00; A61P 3/10

(52) U.S. Cl. 514/613; 435/375; 514/734; 435/29; 506/11; 564/123; 568/729; 800/3

(57) ABSTRACT

Disclosed are compositions and methods related to the ceramide synthesis pathway and various diseases and disorders associated therewith, such as insulin resistance and inflammation.

### CROSS REFERENCE TO RELATED APPLICATIONS

This application claims the benefit of priority of U.S. Provisional Application No. 60/814,932, filed Jun. 19, 2006, which application is incorporated herein by this reference in its entirety.

### ACKNOWLEDGEMENTS

This invention was made with government support under NIH Grant DK58784. The government has certain rights in this invention.

### BACKGROUND

Diabetes mellitus is a major cause of morbidity and mortality. Chronically elevated blood glucose leads to debilitating complications: nephropathy, often necessitating dialysis or renal transplant; peripheral neuropathy; retinopathy leading to blindness; ulceration of the legs and feet, leading to amputation; fatty liver disease, sometimes progressing to cirrhosis; and vulnerability to coronary artery disease and myocardial infarction.

There are two primary types of diabetes. Type I, or insulin-dependent diabetes mellitus (IDDM) is due to autoimmune destruction of insulin-producing beta cells in the pancreatic islets. The onset of this disease is usually in childhood or adolescence. Treatment consists primarily of multiple daily injections of insulin, combined with frequent testing of blood glucose levels to guide adjustment of insulin doses, because excess insulin can cause hypoglycemia and consequent impairment of brain and other functions. Type II, or noninsulin-dependent diabetes mellitus (NIDDM) typically develops in adulthood. NIDDM is associated with resistance of glucose-utilizing tissues like adipose tissue, muscle, and liver, to the actions of insulin. Initially, the pancreatic islet beta cells compensate by secreting excess insulin. Eventual islet failure results in decompensation and chronic hyperglycemia. Conversely, moderate islet insufficiency can precede or coincide with peripheral insulin resistance.

Insulin resistance can also occur without marked hyperglycemia, and is generally associated with atherosclerosis, obesity, hyperlipidemia, and essential hypertension. This cluster of abnormalities constitutes the “metabolic syndrome” or “insulin resistance syndrome”. Insulin resistance is also associated with fatty liver, which can progress to chronic inflammation (NASH; “nonalcoholic steatohepatitis”), fibrosis, and cirrhosis. Cumulatively, insulin resistance syndromes, including but not limited to diabetes, underlie many of the major causes of morbidity and death of people over age 40.

Despite the existence of drugs to treat such disorders, diabetes and other insulin-resistant disorders remain a major and growing public health problem. Late stage complications of diabetes consume a large proportion of national health care resources. There is a need for new orally active therapeutic agents which effectively address the primary defects of insulin resistance and islet failure with fewer or milder side effects than existing drugs. What is needed in the art are compositions and methods for treating insulin resistance.

### SUMMARY

Disclosed herein are methods of modulating insulin resistance in a cell, comprising identifying a cell in need of modulated insulin resistance, and administering to the cell a composition which inhibits ceramide synthesis, thereby modulating insulin resistance in a cell.

Also disclosed are methods of modulating inflammation in a subject, comprising identifying a cell in need of modulated inflammation, and administering to the cell a composition which inhibits ceramide synthesis, thereby modulating inflammation in a cell.

Also disclosed are methods of screening for a test compound that modulates ceramide synthesis comprising: contacting a cell that produces ceramide with a test compound; and detecting altered levels of ceramide synthesis; wherein altered levels of ceramide synthesis indicate a compound that modulates ceramide synthesis.

Also disclosed are methods of screening for a test compound that modulates ceramide synthesis comprising: contacting a transgenic animal that is deficient in one or more of the following proteins: serine palmitoyl transferase, 3-ketosphingamine reductase, dihydroceramide synthase, dihydroceramide desaturase, GlcCer synthase, sphingosine-1-phosphate lyase, sphingosine-phosphate-phosphatase, SM synthase, and sphingomyelinase with a test compound; and detecting a difference in ceramide synthesis in the transgenic animal; wherein a difference in ceramide synthesis indicates a test compound that modulates ceramide synthesis.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows ceramide is a requisite intermediate linking dexamethasone to insulin resistance. (A) Sprague-Dawley rats were treated with myriocin (300 μg/Kg, IP, solid bars) or saline (open bars) for two weeks prior to giving administering dexamethasone (40 μg/Kg, IP) or vehicle (5% ethanol) every 12 hours for 36 hours. Animals were euthanized by CO₂ inhalation, livers were removed, and ceramide levels were assessed as described in SOM-Methods. Shown are mean ceramide levels+/−S.E.M, n=6 per group. The asterisk denotes that the sample was statistically different from vehicle-treated controls, p<0.05, student's t-test. (B) Livers were obtained as in A, above, and protein and mRNA were extracted as described in SOM-Methods. Levels of transcripts (solid bars) encoding serine palimitoyl transferase isoforms 1 and 2 (SPT1 and SPT2), dihydroceramide synthases (DCS) TL1, UOG, and TRH3, glucosylceramide synthase (GCS), and acid ceramidase (AC) transcripts were obtained by reverse-transcriptase polymerase chain reaction, normalized to β-actin. SPT2 protein levels (white bars) were detected by immunoblotting with an antibody from Cayman Chemicals (Ann Arbor, Mich.) and quantified on a Kodak Image Station 2000R (Rochester, N.Y.). Data represent the fold-change in transcript or protein in dexamethasone-treated animals relative to vehicle controls. SPT2, DCS-TL1, DCS-UOG, GCS, and AC were significantly elevated in the dexamethasone-treated animals (n=5, p<0.01). (C and D) Rats were challenged with glucose (1 g/kg) given intraperitoneally (IP) following treatment with dexamethasone (squares), ethanol carrier (triangles), myriocin (open shapes), or saline (solid shapes) as in A, above. Tail vein blood (3 μl) was sampled for glucose (C)(using the Glucometer Elite from Bayer Corp., Tarrytown, N.Y.) or insulin (D) (using an Elisa kit from Linco Diagnostics, Springfield, Mo.). Myriocin significantly lowered the area under the curve for both tests (n=6 per group, p<0.05). (E-G) Hyperinsulinemic euglycemic clamps were performed on animals (n=4 per group) treated as in A, above, as described in SOM-Methods. Data from animals receiving myriocin are depicted in solid bars. (E) The glucose infusion rate required to maintain normal glycemia during insulin infusion (4mU/kg/min) was calculated as the average infusion rate during the 60 minute steady state. (F) Hepatic glucose output was estimated by the disappearance of [¹⁴C] glucose from a bolus dose given during the steady state minus the glucose infusion rate. (G) Disappearance of a bolus dose of [³H] 2-deoxyglucose from the bloodstream was used to estimate the rate of whole body glucose uptake. In E-G, data presented as the mean+/−the S.E.M In all cases, myriocin significantly restored insulin-responsiveness (p<0.05, student's t-test).

FIG. 2 shows that mice lacking dihydroceramide desaturase 1 (Des1) are resistant to dexamethasone-induced insulin resistance. (A) Mice lacking DES1 were generated as described in SOM-Methods. Mice lacking one allele DES1^{-/+} were indistinguishable from wild-type DES 1^{+/+} animals, while those lacking both alleles DES1^{-/-}-ones had a markedly different phenotype that is described in the text. (B) Whole body ceramide and dihydroceramide were quantified from day old DES 1^{+/+} (open bars), DES1^{+/-} (hatched bars), or Des1^{-/-} (solid bars) mice (n=3) by the Lipidomics core at the Medical University of South Carolina. (C) Glucose tolerance tests were performed on 3-7 week old DES1^{+/+} (squares), DES1^{+/-} (triangles), or DES1^{-/-} (circles) mice. Data are reported as the mean+/−the S.E.M. [n=7 (+/+), n=12 (−/+), n=3 (−/−)]. (D) Ceramide and dihycroceramide were quantified as in B, above, from individual tissues isolated from 7-week old DES 1^{+/+} (open bars) or DES1^{+/-} (solid bars) mice. The data are presented as the mean ratio of ceramide/dihydroceramide+/−S.E.M. (E) Fasting insulin resistance indices were calculated (glucose concentration [mmol/L)×insulin concentration (mU/L)÷22.5] from mice treated for 1 week with dexamethasone (2 mg/kg/day, solid bars) or vehicle (open bars). Data are presented as mean+/−S.E.M. (n=4 per group) (F) Insulin tolerance tests were initiated by insulin injection (0.75 mU/kg, IP) into DES1^{+/+} (squares) or DES1^{+/-} (triangles) mice treated with dexamethasone (dashed lines, filled shapes) or carrier (solid lines, open shapes), with glucose values obtained at the indicated time point using a glucometer. Data are presented as the mean glucose level normalized to the fasting glucose concentration+/−the S.E.M. (n=5, * p<0.05, †p<0.05, denotes difference from dexamethasone treated groups).

FIG. 3 shows ceramide is requisite for saturated fat, but not unsaturated fat, induced insulin resistance. A-B Glycerol (hatched bars), lard oil (solid bars) or soy oil (open bars) were intravenously infused into rats for 6 hours in the presence or absence of myriocin (MYR) or L-cycloserine (CS). Following infusion, rats were euthanized with pentobarbital and soleus muscles were rapidly dissected and frozen in liquid nitrogen. Ceramide (A) and DAG (B) levels were quantified (n=6 per group) (C). Hyperinsulinemic euglycemic clamps were initiated after 4.5 hours of infusion. Depicted is the mean glucose infusion rate required to maintain steady-state euglycemia for 30 minutes+/−S.E.M. (n=6 per group). (D-F) Soleus muscles were isolated, bisected and placed in oxygenated Krebs-Hensliet buffer containing 2.5% FFA free bovine serum albumin (BSA, hatched bars), 1 mM palmitate (solid bars) or 1 mM linoleate (open bars). Indicated muscle strips were maintained in the presence of cycloserine (CS, 1 mM) or myriocin (MYR, 10 μM) throughout the experiments. (D) Ceramide and (E) diacyglycerol were quantified data were normalized to the BSA treated muscle strip from the same rat. (A) 2-deoxyglucose uptake was measured in the absence (open bars) or presence of insulin (300 μU/mL, solid bars) during the final 20 minutes of the 6 hour incubation. Data presented as the mean+/−the S.E.M. (n=6 per group, p<0.05 by students' t-test). (G) Intact soleus muscles were dissected from 15 to 20 week-old DES1^{-/+} and DES1^{+/+} mice and incubated for 6 hours in lipid as indicated. During the final hour of the incubation, muscles were treated with or without insulin (300 μU/ml) as indicated in the SOM-Methods section. 2-Deoxyglucose uptake was assessed as in F, above. Data are presented as the mean+/−S.E.M., n=6.

FIG. 4 shows myriocin improves glucose homeostasis and prevents diabetes in ZDF rats. Myriocin administered by intraperitoneal injection (0.3 mg/kg, IP, every other day) markedly improved glucose homeostasis in male ZDF rats. 7 week-old animals were pre-bled and assigned to vehicle (1% w/v carboxymethylcellulose, 0.25% Tween-80, open bars) or myriocin (oral gavage) groups based on starting plasma glucose levels and body weight. Blood samples were obtained 1 hour post-dose at the indicated days from the tail vein of conscious animals by gentle massage following tail-snip. Plasma was used for measurements of glucose (Hitachi 911 clinical chemistry analyzer, Roche, Indianapolis, Ind.). Data are presented as the mean+/−the S.E.M. (n=4 per group).

FIG. 5 shows the schematic diagram depicting reactions controlling ceramide synthesis and metabolism. The initial, rate-limiting step in sphingolipid biosynthesis involves the condensation of serine and palmitoyl-CoA, a reaction catalyzed by serine palmitoyltransferase (SPT). Several dihydroceramide synthase isoforms catalyze the addition of a second fatty acyl chain of varying length. Synthesis of biologically active ceramide is completed by dihydroceramide desaturase (Des1). Inhibitors of de novo ceramide synthesis used in the current study are indicated (red).

FIG. 6 shows (A) insulin tolerance tests were performed on male Sprague Dawley Rats (˜250 g) treated with dexamethasone (400 μg/kg, IP, 24 and 12 hours prior to study, squares) or carrier (5% ethanol, triangles) following 1 week of treatment with myriocin (0.3 mg/kg/2 days, dashed lines, open shapes) or saline (solid lines, solid shapes). Following an overnight fast, insulin was injected (20 U, IP) and whole blood glucose was determined by glucometer. Values are presented as the ratio of measured blood glucose to fasting blood glucose. Data presented as the mean+/−the S.E.M. (n=6 per group, p<0.05 by students' t-test).

FIG. 7 shows (A) gene trap mutation of the Des-1 gene. SA, splice acceptor sequence. Neo, neomycin resistance gene. (B). Des-1 RT-PCR. Primers A and B are complementary to Des-1 exons 1 and 2 flanking the insertion site of the gene trapping vector. RT-PCR using primers A and B shows absence of endogenous Des-1 message in the kidney and liver of homozygous animals, respectively. RT, reverse transcriptase. (C). Table depicting the distribution of offspring of DES1^{-/+} parents. (D) Glucose tolerance tests were initiated by glucose injection (1 mg/kg, IP) into 3-8 week old DES1^{+/+} (squares), DES1^{-/+} (triangles) and DES1^{-/-} (diamonds) Des1 null mice. Glucose concentrations were assessed by glucometer. [(+/+) n=10 female, 4 male; (−/+) n—12 female, 8 male; (−/−) n=1 male, 1 female).

FIG. 8 shows mass of obese ZDF (solid shapes) and lean control rats (open shapes) treated with myriocin (0.3 mg/kg/2 days, triangles with dashed lines) or saline (squares with solid lines) for 6 weeks.

FIG. 9 shows a schematic depicting the targeting vector for DES1 conditional knockout.

FIG. 10 shows TNFα increases expression of the two subunits of serine glucosylceramide synthase. Using RT-PCR, mRNA levels of serine palmitoyltransferase subunits (LCB1 and LCB2), acid ceramidase (AC), glucosylceramide synthase (GCS), ceramide kinase (CerK), and ceramide synthase isoforms 1-6 (Cers/Lass 1-6) were evaluated.

FIG. 11 shows a schematic depicting the inhibition of Akt/PKB activation by two independent mechanisms.

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

The term “multiwell plate” refers to a two dimensional array of addressable wells located on a substantially flat surface. Multiwell plates can include any number of discrete addressable wells, and include addressable wells of any width or depth. Common examples of multiwell plates include 96 well plates, 384 well plates and 3456 well Nanoplates™. Such multiwell plates can be constructed of plastic, glass, or any essentially electrically nonconductive material

The term “gene knockout” as used herein, refers to the targeted disruption of a gene in vivo with complete loss of function that has been achieved by any transgenic technology familiar to those in the art. In one embodiment, transgenic animals having gene knockouts are those in which the target gene has been rendered nonfunctional by an insertion targeted to the gene to be rendered non-functional by homologous recombination.

The term “hit” refers to a test compound that shows desired properties in an assay.

The term “repetitive” means to repeat at least twice.

The term “test compound” refers to a chemical to be tested by one or more screening method(s) of the invention as a putative modulator. A test compound can be any chemical, such as an inorganic chemical, an organic chemical, a protein, a peptide, a carbohydrate, a lipid, or a combination thereof. Usually, various predetermined concentrations of test compounds are used for screening, such as 0.01 micromolar, 1 micromolar and 10 micromolar. Test compound controls can include the measurement of a signal in the absence of the test compound or comparison to a compound known to modulate the target.

The term “transgenic” is used to describe an organism that includes exogenous genetic material within all of its cells. The term includes any organism whose genome has been altered by in vitro manipulation of the early embryo or fertilized egg or by any transgenic technology to induce a specific gene knockout.

The term “transgene” refers to any piece of DNA which is inserted by artifice into a cell, and becomes part of the genome of the organism (i.e., either stably integrated or as a stable extrachromosomal element) which develops from that cell. Such a transgene can include a gene which is partly or entirely heterologous (i.e., foreign) to the transgenic organism, or may represent a gene homologous to an endogenous gene of the organism. Included within this definition is a transgene created by the providing of an RNA sequence that is transcribed into DNA and then incorporated into the genome. The transgenes of the invention include DNA sequences that encode the fluorescent or bioluminescent protein that may be expressed in a transgenic non-human animal.

The term “activity” as used herein refers to a measurable result of the interaction of molecules. Some exemplary methods of measuring these activities are provided herein.

The term “modulate” as used herein refers to the ability of a compound to change an activity in some measurable way as compared to an appropriate control. As a result of the presence of compounds in the assays, activities can increase (e.g. there could be increased levels of ceramide synthesis), or “decrease” (e.g. there could be decreased levels of ceramide synthesis) as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

The term “monitoring” as used herein refers to any method in the art by which an activity can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “preventing” as used herein refers to administering a compound prior to the onset of clinical symptoms of a disease or conditions so as to prevent a physical manifestation of aberrations associated with the disease or condition.

The term “treating” as used herein refers to administering a compound after the onset of clinical symptoms.

The term “in need of treatment” as used herein refers to a judgment made by a caregiver (e.g. physician, nurse, nurse practitioner, or individual in the case of humans; veterinarian in the case of animals, including non-human mammals) that an individual or animal requires or will benefit from treatment. This judgment is made based on a variety of factors that are in the realm of a care giver's expertise, but that include the knowledge that the individual or animal is ill, or will be ill, as the result of a condition that is treatable by the compounds of the invention.

The term “individual” as used herein refers to a mammal, including animals, preferably mice, rats, other rodents, rabbits, dogs, cats, swine, cattle, sheep, horses, or primates, most preferably humans.

The term “non-human animal” refers to any non-human vertebrate, birds and more usually mammals, preferably primates, animals such as swine, goats, sheep, donkeys, horses, cats, dogs, rabbits or rodents, more preferably rats or mice. Both the terms “animal” and “mammal” expressly embrace human subjects unless preceded with the term “non-human”.

The terms “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. GENERAL

Insulin resistance is an underlying feature of both type 2 diabetes (T2D) and the metabolic syndrome. The sphingolipid ceramide is a common molecular intermediate linking several different pathological metabolic stresses to the induction of insulin resistance. Moreover, inhibition of ceramide synthesis markedly improved glucose tolerance and prevented the onset of frank diabetes in obese rodents. Collectively these data identify enzymes required for de novo ceramide synthesis as novel therapeutic targets for combating insulin resistance caused by nutrient excess or glucocorticoid therapy.

Insulin resistance is prevalent in 20-25% of the population, and the condition is a chief component of Type 2 Diabetes Mellitus and a risk factor for cardiovascular disease and certain forms of cancer (Reaven 2005). Obesity predisposes individuals to the development of insulin resistance, and several mechanisms have been proposed to explain how increased adiposity antagonizes insulin-stimulation of nutrient uptake and storage. In some obese individuals, increased adipose tissue mass may trigger the synthesis and/or secretion of glucocorticoids (Hermanowski-Vosatka, 2005) or inflammatory cytokines (e.g. tumor necrosis factor alpha) (Hotamisligil 2005) which inhibit insulin action in peripheral tissues. This can occur through TNF-induced expression of certain ceramide synthase isoforms (see Example 3). Additionally, excess lipids may be delivered to non-adipose tissues which are not suited for fat storage (i.e. skeletal muscle and the liver), thus leading to the formation of specific metabolites that directly antagonize insulin signaling and action (Schmitz-Peiffer 2000, McGarry 2002).

The stresses associated with obesity almost invariably generate ceramide (Summers 2005). Moreover, studies in cultured cells suggest that the sphingolipid and its derivatives antagonize insulin-stimulation of glucose uptake and storage, and thus can initiate many of the molecular defects which underlie insulin resistance. First, through its targets protein phosphatase 2A and protein kinase Cζ (Wang 1998, Summers 1998), ceramide inhibits insulin-stimulation of Akt/Protein Kinase B, a central mediator of glucose transport and anabolic metabolism (Chavez 2003, Chavez 2005, Powell 2003, Powell 2004). Second, ceramide activates IκKβ and JNK (Ruvulo 2003), which alter gene expression patterns to promote insulin resistance (Yuan 2001, Hirosumi 2002). Third, ceramide inhibits components of the electron transport chain (Gudz 1997) and alters mitochondrial membrane permeability (Siskind 2002), and thus may contribute to mitochondrial dysfunction supporting oxidative stress and diabetes (Houstis 2006, Powell 2005). And fourth, ceramide derivatives such as sphingosine (Nelson 1986) and ganglioside GM3 (Tagami 2002) inhibit various steps in insulin-signal transduction. The inhibition of ceramide synthesis can combat several underlying causes of insulin resistance, and thus improve insulin sensitivity in tissues exposed to multiple different metabolic insults.

Synthetic glucocorticoids, one of the most frequently prescribed classes of therapeutics, impair glucose tolerance. This is particularly troublesome given the large number of insulin resistant or diabetic individuals receiving these drugs. Though obese individuals do not have elevated glucocorticoid levels in the circulation, increased intracellular glucocorticoid tone, likely driven by increased activity of 11β-hydroxysteroid dehydrogenase type 1 which converts inactive cortisone into active cortisol, causes insulin resistance in rodents and has been proposed to mediate the development of the disease in humans (Masuzaki 2001, Masuzaki 2003, Kotelevtsev 1999, Kotelevtsev 1997). Herein it is demonstrated that ceramide synthesis is requisite for glucocorticoid-induced insulin resistance.

Disclosed herein are methods of modulating insulin resistance in a cell, comprising identifying a cell in need of modulated insulin resistance, and administering to the cell a composition which inhibits ceramide synthesis, thereby modulating insulin resistance in a cell. As described below, the cell can be in vitro, in vivo, or ex vivo. When the cell is in a subject, the subject can have any one or more of the following diseases and disorders: metabolic syndrome, obesity, diabetes (such as Type II), or Cushing's disease. The subject can also have inflammation. The subject can also have Gaucher disease. These diseases and disorders, as well as others, are disclosed in more detail below.

Ceramide synthesis is a complex pathway involving many components. Specifically, although sphingolipids represent a significant dietary component, they are largely degraded in the mammalian intestine, and their production in animal tissues is primarily dependent on a widespread biosynthetic pathway. The initial, rate-limiting reaction is the condensation of palmitoyl CoA and serine, a reaction is catalyzed by serine palmitoyltransferase (SPT), to produce 3-oxosphinganine. The availability of palmitoyl-CoA and serine strongly influences the rate of this reaction. Three reactions follow, resulting in sphinganine→dihydroceramide→ceramide production. Once generated, ceramide is a basic building block for numerous additional sphingolipid derivatives including sphingomyelin, sphingosine 1-phosphate (S1P), ceramide 1-phosphate, and a large family of glucosylceramides. Proteins involved in this pathway include, but are not limited to, serine palmitoyl transferase, 3-ketosphingamine reductase, dihydroceramide synthase, dihydroceramide desaturase, GlcCer synthase, sphingosine-1-phosphate lyase, sphingosine-phosphate-phosphatase, SM synthase, and sphingomyelinase. Therefore, inhibiting any of the components of this pathway can inhibit ceramide synthesis, and thereby modulate insulin resistance as well as the other diseases and disorders mentioned herein. Particularly, the enzyme dihydroceramide desaturase can be inhibited. An example of a compound that can inhibit dihydroceramide desaturase includes GT-11.

As disclosed herein, dihydroceramide desaturase 1 (DES1) is part of the ceramide synthesis pathway. It has been shown that both of the drugs fenretinide and resveratrol can be used to inhibit DES 1, thereby inhibiting ceramide synthesis. (Zheng et al. Biochim Biophys Acta. 2006 December; 1758(12):1864-84, herein incorporated by reference in its entirety for its teaching concerning DES 1 and fenretinide; and Szkudelski et al, European Journal of Pharmacology Volume 552, Issues 1-3, 15 Dec. 2006, Pages 176-181, herein incorporated by reference in its entirety for its teaching concerning insulin and resveratrol). Therefore, disclosed herein is a method of treating a disease associated with ceramide comprising administering a ceramide synthesis inhibitor to the subject in need thereof. In one example, the ceramide synthesis inhibitor is resveratrol or fenretinide.

Metabolic Syndrome (also known as Syndrome X) is characterized by having at least three of the following symptoms: insulin resistance; abdominal fat—in men this is defined as a 40 inch waist or larger, in women 35 inches or larger; high blood sugar levels—at least 110 milligrams per deciliter (mg/dL) after fasting; high triglycerides—at least 150 mg/dL in the blood stream; low HDL—less than 40 mg/dL; pro-thrombotic state (e.g. high fibrinogen or plasminogen activator inhibitor in the blood); or blood pressure of 130/85 mmHg or higher. A connection has been found between Metabolic Syndrome and other conditions such as obesity, high blood pressure and high levels of LDL “bad” cholesterol, all of which are risk factors for Cardiovascular Disease. For example, an increased link between Metabolic Syndrome and atherosclerosis has been shown. People with Metabolic Syndrome are also more prone to developing Type 2 Diabetes, as well as PCOS (Polycystic Ovarian Syndrome) in women and prostate cancer in men.

As described above, insulin resistance can be manifested in several ways, including Type 2 Diabetes. Type 2 diabetes is the condition most obviously linked to insulin resistance. Compensatory hyperinsulinemia helps maintain normal glucose levels—often for decades—before overt diabetes develops. Eventually the beta cells of the pancreas are unable to overcome insulin resistance through hypersecretion. Glucose levels rise, and a diagnosis of diabetes can be made. Patients with type 2 diabetes remain hyperinsulinemic until they are in an advanced stage of disease.

Insulin resistance can also include hypertension. One half of patients with essential hypertension are insulin resistant and hyperinsulinemic. There is evidence that blood pressure is linked to the degree of insulin resistance.

Hyperlipidemia is also associated with insulin resistance. The lipid profile of patients with type 2 diabetes includes decreased high-density lipoprotein cholesterol levels (a significant risk factor for heart disease), increased serum very-low-density lipoprotein cholesterol and triglyceride levels and, sometimes, a decreased low-density lipoprotein cholesterol level. Insulin resistance has been found in persons with low levels of high-density lipoprotein. Insulin levels have also been linked to very-low-density lipoprotein synthesis and plasma triglyceride levels.

Atherosclerotic heart disease is also associated with insulin resistance, as is obesity. Many persons with one or more of the conditions listed above are obese. Obesity is a component of the syndrome, but it promotes insulin resistance rather than resulting from it.

Other abnormalities linked to insulin resistance include hyperuricemia, elevated levels of plasminogen activator inhibitor 1 and a preponderance of small-size, low-density lipoprotein particles. Higher plasminogen activator inhibitor 1 levels and decreased low-density lipoprotein particle diameter are thought to increase the risk of coronary heart disease.

Inhibition of ceramide synthesis can be used to treat inflammation.

Inflammation is a complex stereotypical reaction of the body expressing the response to damage of its cells and vascularized tissues. The discovery of the detailed processes of inflammation has revealed a close relationship between inflammation and the immune response. The main features of the inflammatory response are vasodilation, i.e. widening of the blood vessels to increase the blood flow to the infected area; increased vascular permeability, which allows diffusible components to enter the site; cellular infiltration by chemotaxis, or the directed movement of inflammatory cells through the walls of blood vessels into the site of injury; changes in biosynthetic, metabolic, and catabolic profiles of many organs; and activation of cells of the immune system as well as of complex enzymatic systems of blood plasma.

There are two forms of inflammation, acute and chronic. Acute inflammation can be divided into several phases. The earliest, gross event of an inflammatory response is temporary vasoconstriction, i.e. narrowing of blood vessels caused by contraction of smooth muscle in the vessel walls, which can be seen as blanching (whitening) of the skin. This is followed by several phases that occur over minutes, hours and days later. The first is the acute vascular response, which follows within seconds of the tissue injury and lasts for several minutes. This results from vasodilation and increased capillary permeability due to alterations in the vascular endothelium, which leads to increased blood flow (hyperemia) that causes redness (erythema) and the entry of fluid into the tissues (edema).

This can be followed by an acute cellular response, which takes place over the next few hours. The hallmark of this phase is the appearance of granulocytes, particularly neutrophils, in the tissues. These cells first attach themselves to the endothelial cells within the blood vessels (margination) and then cross into the surrounding tissue (diapedesis). During this phase erythrocytes may also leak into the tissues and a hemorrhage can occur. If the vessel is damaged, fibrinogen and fibronectin are deposited at the site of injury, platelets aggregate and become activated, and the red cells stack together in what are called “rouleau” to help stop bleeding and aid clot formation. The dead and dying cells contribute to pus formation. If the damage is sufficiently severe, a chronic cellular response may follow over the next few days. A characteristic of this phase of inflammation is the appearance of a mononuclear cell infiltrate composed of macrophages and lymphocytes. The macrophages are involved in microbial killing, in clearing up cellular and tissue debris, and in remodeling of tissues.

Chronic inflammation is an inflammatory response of prolonged duration—weeks, months, or even indefinitely—whose extended time course is provoked by persistence of the causative stimulus to inflammation in the tissue. The inflammatory process inevitably causes tissue damage and is accompanied by simultaneous attempts at healing and repair. The exact nature, extent and time course of chronic inflammation is variable, and depends on a balance between the causative agent and the attempts of the body to remove it. Etiological agents producing chronic inflammation include: (i) infectious organisms that can avoid or resist host defenses and so persist in the tissue for a prolonged period. Examples include Mycobacterium tuberculosis, Actinomycetes, and numerous fungi, protozoa and metazoal parasites. Such organisms are in general able to avoid phagocytosis or survive within phagocytic cells, and tend not to produce toxins causing acute tissue damage. (ii) Infectious organisms that are not innately resistant but persist in damaged regions where they are protected from host defenses. An example is bacteria which grow in the pus within an undrained abscess cavity, where they are protected both from host immunity and from blood-borne therapeutic agents, e.g. antibiotics. Some locations are particularly prone to chronic abscess formation, e.g. bone, and pleural cavities. (iii) Irritant non-living foreign material that cannot be removed by enzymatic breakdown or phagocytosis. Examples include a wide range of materials implanted into wounds (wood splinters, grit, metals and plastics), inhaled (silica dust and other particles or fibers), or deliberately introduced (surgical prostheses, sutures, etc.) Also included are transplants. Dead tissue components that cannot be broken down may have similar effects, e.g. keratin squames from a ruptured epidermoid cyst or fragments of dead bone (sequestrum) in osteomyclitis. (iv) In some cases the stimulus to chronic inflammation may be a normal tissue component. This occurs in inflammatory diseases where the disease process is initiated and maintained because of an abnormality in the regulation of the body's immune response to its own tissues—the so-called auto-immune diseases. (v) For many diseases characterized by a chronic inflammatory pathological process the underlying cause remains unknown. A good example is Crohn's disease of the intestine.

Examples of chronic inflammatory diseases include tuberculosis, chronic cholecystitis, bronchiectasis, rheumatoid arthritis, Hashimoto's thyroiditis, inflammatory bowel disease (ulcerative colitis and Crohn's disease), silicosis and other pneumoconiosis, and implanted foreign body in a wound.

Inhibitors of ceramide synthesis can also be used to prevent infection. Sphingolipids have been shown to render one susceptible to infection by either facilitating the entry of viruses or bacteria into host cells or by decreasing an individual's resistance to these pathogens. Due to their long, largely saturated acyl chains, sphingolipids tend to pack together in microdomains that exclude phospholipids. In the presence of cholesterol, these sphingolipids organize themselves in raft structures that can be isolated from other membrane fractions due to their insolubility to some non-ionic detergents. Ceramide, because of its tendency to self-associate, induces the coalescence of microscopic rafts into large-membrane macrodomains. Raft domains have been shown to recruit certain types of cellular proteins, while excluding others, and are important for processes such as signal transduction, sorting, and endocytosis. It has been shown that these raft structures facilitate entry of various pathogens. The importance of ceramides in pathogen entry is underscored in studies looking at Neisseria gonorrhoeae, Pseudomonas aeruginosa, Staphylococcus aureus, and Sindbis virus, which have been shown to activate acid sphingomyelinase to rapidly induce ceramide formation. A strength of these studies was the observation that inactivation of acid sphingomyelinase greatly hindered pathogen internalization. In addition, the protozoan, Leishmania donovani, was shown to induce ceramide generation through both de novo synthesis and activation of sphingomyelinase, and the elevated ceramide was shown to facilitate the survival of leishmanial parasites in the intramacrophageal milieu. Rafts, in addition to playing a potential role in pathogen entry, have been shown to serve as platforms for viral assembly or budding.

The susceptibility to infection for individuals with these diseases additionally involves an altered immune response, which renders them susceptible to opportunistic pathogens. For example, uncontrolled diabetes demonstrate defective migration of polymorphonuclear leukocytes, which ingest and destroy microbes, as well as impaired phagocytosis of the invading pathogen. Similarly, glucocorticoids have been shown to inhibit superoxide production, which is important for the destruction of the invading agent, both in vitro and in vivo. Ceramides have been shown to mimic these effects as well. For example, increasing endogenous ceramide levels to a maximal level terminates functional responses in polymorphonuclear leukocytes, as ceramide inhibits phagocytosis and blocks superoxide release. Diabetics additionally exhibit an exaggerated inflammatory response to microbial products, which further compromises healing, and ceramides or sphongosine have been shown to augment the inflammatory response of TNFα or other pro-inflammatory cytokines. In addition, ceramides and other sphingolipids have been shown to positively or negatively affect the function of mononuclear phagocytes, mast cells, dendritic cells, natural killer cells, cytotoxic T lymphocytes, B lymphocytes, and others. One can easily envision how globally altering sphingolipid levels could interfere with the development of an appropriate defense against invading pathogens.

Modulation of ceramide synthesis can also be used to treat Gaucher Disease. Gaucher disease is a genetic disorder caused by deficiency of the enzyme glucocerebrosidase. This deficiency causes a fatty substance to accumulate in certain body tissues such as the spleen, liver, and bone marrow. There are three types of Gaucher disease: Type I and Type II. Symptoms of Gaucher disease usually become apparent in childhood or early adulthood but can be diagnosed at anytime throughout someone's life. An individual affected by Gaucher disease might exhibit one or several of these symptoms: enlarged liver and/or spleen, anemia, fatigue, easy bruising, impaired blood clotting (e.g. frequent nose bleeds without trauma and gum bleeding with regular brushing, bruising for no reason), bone pain, and fractures

Also disclosed are method of screening for a test compound that modulates ceramide synthesis comprising contacting a cell that produces ceramide with a test compound; and detecting altered levels of ceramide synthesis; wherein altered levels of ceramide synthesis indicate a compound that modulates ceramide synthesis. The compounds can be in a high throughput system, as described below. Also disclosed are compounds identified by the screening methods disclosed herein.

Also disclosed are in vivo methods of screening for a test compound that modulates ceramide synthesis comprising: contacting a transgenic animal that is deficient in one or more of the following proteins: serine palmitoyl transferase, 3-ketosphingamine reductase, dihydroceramide synthase, dihydroceramide desaturase, GlcCer synthase, sphingosine-1-phosphate lyase, sphingosine-phosphate-phosphatase, SM synthase, and sphingomyelinase with a test compound; and detecting a difference in ceramide synthesis in the transgenic animal; wherein a difference in ceramide synthesis indicates a test compound that modulates ceramide synthesis. Also disclosed are compounds detected by the in vivo screening methods disclosed herein.

### C. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example Genbank No. NM_{—}003676 sets forth a particular sequence of the protein encoded by SEQ ID NO: 1. Serine palmitoylstransferase is shown in Genbank Nos NM_{—}006415 (SPTLC1) and NM_{—}178324 (SPTLC2), Dihydroceramide synthase is shown in Genbank Nos. AU080088 (LASS1 (UOG)) and AU080131 (MNCb-5211). Genbank No. NM_{—}024552 (LASS4 (TRH)=, LASS5=NM_{—}147190, LASS 6=NM_{—}203463, LASS 6=NM_{—}203463, LASS2=NM_{—}181746, LASS3=BC027616)). Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Homology/Identity

It is understood that one way to define any known variants and derivatives or those that might arise, of the disclosed genes and proteins herein is through defining the variants and derivatives in terms of homology to specific known sequences. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al., Proc. Natl. Acad. Sci. USA 86:7706-7710, 1989, Jaeger et al. Methods Enzymol. 183:281-306, 1989 which are herein incorporated by reference for at least material related to nucleic acid alignment.

2. Nucleic Acids

The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with any of the components of ceramide synthesis, such as serine palmitoyl transferase, 3-ketosphingamine reductase, dihydroceramide synthase, dihydroceramide desaturase, GlcCer synthase, sphingosine-1-phosphate lyase, sphingosine-phosphate-phosphatase, SM synthase, and sphingomyelinase. Examples of the components of ceramide synthesis can be found, for example, in Summers et al., Diabetes Vol. 54, March 2005, pages 591-602, herein incorporated by reference in its entirety for its teaching concerning the ceramide synthase pathway. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁴, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide.

Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al. Proc. Natl. Acad. Sci. (USA) 92:2627-2631 (1995)). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162.

3. Nucleic Acid Delivery

In the methods described herein which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

4. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as a ceramide synthase inhibitor, into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B 19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpes virus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed nucleic acids or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother. 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research. 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

5. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banexji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

6. Peptides

a) Protein Variants

As discussed herein there are numerous variants of the components of the ceramide synthesis pathway that are known and herein contemplated. Examples include, but are not limited to, serine palmitoyl transferase, 3-ketosphingamine reductase, dihydroceramide synthase, dihydroceramide desaturase, GlcCer synthase, sphingosine-1-phosphate lyase, sphingosine-phosphate-phosphatase, SM synthase, and sphingomyelinase. In addition, to the known functional strain variants there are derivatives of these proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| Alanine | Ala (A) |
| Allosoleucine | AIle |
| Arginine | Arg (R) |
| Asparagines | Asn (N) |
| aspartic acid | Asp (D) |
| Cysteine | Cys (C) |
| glutamic acid | Glu (E) |
| Glutamine | Gln (K) |
| Glycine | Gly (G) |
| Histidine | His (H) |
| Isolelucine | Ile (I) |
| Leucine | Leu (L) |
| Lysine | Lys (K) |
| Phenylalanine | Phe (F) |
| Praline | Pro (P) |
| pyroglutamic acid | PGlu |
| Serine | Ser (S} |
| Threonine | Thr (T) |
| Tyrosine | Tyr (Y) |
| Tryptophan | Trp (W) |
| Valine | Val (V) |

TABLE 2
Amino Acid Substitutions
Original Residue Exemplary Conservative Substitutions, others are
known in the art.

Ala; ser
Arg; lys, gln
Asn; gln; his
Asp; glu
Cys; ser
Gln; asn, lys
Glu; asp
Gly; pro
His; asn; gln
Ile; leu; val
Leu; ile; val
Lys; arg; gln
Met; leu; ile
Phe; met; leu; tyr
Ser; thr
Thr; ser
Trp; tyr
Tyr; trp; phe
Val; ile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 1 sets forth a particular sequence of DES-1 and SEQ ID NO: 2 sets forth a particular sequence of a nucleic acid encoding DES-1. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂S (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of IL-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

7. Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with components of the ceramide synthesis pathway. As discussed above, examples of enzymes that are part of this pathway include, but are not limited to, serine palmitoyl transferase, 3-ketosphingamine reductase, dihydroceramide synthase, dihydroceramide desaturase, GlcCer synthase, sphingosine-1-phosphate lyase, sphingosine-phosphate-phosphatase, SM synthase, and sphingomyelinase. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing anti DES-1 antibodies, for example, and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

8. Pharmaceutical carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing insulin resistance, cancer, or other diseases or disorders, the efficacy of the therapeutic antibody can be assessed in various ways well known to the skilled practitioner.

The compositions that inhibits ceramide synthesis, or reduces ceramide production, disclosed herein may be administered prophylactically to patients or subjects who are at risk for insulin resistance, or metabolic syndrome. Other molecules that interact with ceramide but which do not have a specific pharmaceutical function, but which may be used for tracking changes within cellular chromosomes or for the delivery of diagnostic tools for example can be delivered in ways similar to those described for the pharmaceutical products.

The disclosed compositions and methods can also be used for example as tools to isolate and test new drug candidates for a variety of insulin and metabolic-related diseases.

9. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

10. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

11. Compositions Identified by Screening with Disclosed Compositions/Combinatorial Chemistry

a) Combinatorial Chemistry

The disclosed compositions can be used as targets for any combinatorial technique to identify molecules or macromolecular molecules that interact with the disclosed compositions in a desired way. Also disclosed are the compositions that are identified through combinatorial techniques or screening techniques in which the compositions disclosed herein, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, those that interact with DES-1 or other components of the ceramide synthesis pathway, such as serine palmitoyl transferase, 3-ketosphingamine reductase, dihydroceramide synthase, dihydroceramide desaturase (DES-1), GlcCer synthase, sphingosine-1-phosphate lyase, sphingosine-phosphate-phosphatase, SM synthase, and sphingomyelinase, are also disclosed. Thus, the products produced using the combinatorial or screening approaches that involve the disclosed compositions, such as DES-1, are also considered herein disclosed.

It is understood that the disclosed methods for identifying molecules that interact with components of the ceramide synthesis pathway, for example, can be performed using high through put means. For example, putative inhibitors can be identified using Fluorescence Resonance Energy Transfer (FRET) to quickly identify interactions. The underlying theory of the techniques is that when two molecules are close in space, i.e., interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptidyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein: protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example an extracellular portion of DES-1 is attached to a DNA binding domain of a transcriptional activation protein, such as Gal 4. By performing the Two-hybrid technique on this type of system, molecules that bind the extracellular portion of DES-1 can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in iterative processes.

12. Screening Methods

Disclosed herein is a method of screening for a test compound that modulates ceramide synthesis comprising: contacting a cell that produces ceramide with a test compound; and detecting altered levels of ceramide synthesis; wherein altered levels of ceramide synthesis indicate a compound that modulates ceramide synthesis.

Also disclosed is a method of screening for a test compound that modulates ceramide synthesis comprising: contacting a transgenic animal that is deficient in one or more of the following proteins: serine palmitoyl transferase, 3-ketosphingamine reductase, dihydroceramide synthase, dihydroceramide desaturase, GlcCer synthase, sphingosine-1-phosphate lyase, sphingosine-phosphate-phosphatase, SM synthase, and sphingomyelinase with a test compound; and detecting a difference in ceramide synthesis in the transgenic animal; wherein a difference in ceramide synthesis indicates a test compound that modulates ceramide synthesis.

The modulation can comprise an increase in ceramide synthesis or related activity. By an “increase” is meant that the activity is greater in the presence of the test compound than not in the presence of the test compound. The modulation can also comprise a decrease in ceramide synthesis or downstream activity. By a “decrease” is meant that the activity is less in the presence of the test compound than not in the presence of the test compound.

The response of ceramide synthesis can be measured in the presence of various concentrations of test compound. The measuring steps can also comprise measuring the response at various concentrations of the test compound. For example, the concentration of the test compound can range from 1 nM to 1000 μM.

Assays contemplated by the invention include both binding assays and activity assays; these assays may be performed in conventional or high throughput formats. Modulator screens are designed to identify stimulatory and inhibitory agents. The sources for potential agents to be screened include natural sources, such as a cell extract (e.g., invertebrate cells including, but not limited to, bacterial, fungal, algal, and plant cells) and synthetic sources, such as chemical compound libraries or biological libraries such as antibody substance or peptide libraries. Agents are screened for the ability to either stimulate or inhibit the activity. Binding assays are used to detect activity levels. Both functional and binding assays of activity are readily adapted to screens for modulators such as agonist (stimulatory) and antagonist (inhibitory) compounds.

Contemplated herein are a multitude of assays to screen and identify modulators, such as agonists and antagonists, of ceramide synthesis (and downstream activity). In one example, the cell is immobilized and interaction with a candidate modulator is detected. In another example, the test compound is immobilized. In yet another example, interaction between ceramide and the test compound is assessed in a solution assay. Another contemplated assay involves a variation of the di-hybrid assay wherein a modulator of protein/protein interactions is identified by detection of a positive signal in a transformed or transfected host cell.

Candidate modulators for screening according to contemplated by the invention include any chemical compounds, including libraries of chemical compounds. There are a number of different libraries used for the identification of small molecule modulators, including: (1) chemical libraries, (2) natural product libraries, and (3) combinatorial libraries comprised of random peptides, oligonucleotides or organic molecules. Chemical libraries consist of random chemical structures, or analogs of known compounds, or analogs of compounds that have been identified as “hits” or “leads” in prior drug discovery screens, some of which may be derived from natural products or from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282:63-68 (1998). Combinatorial libraries are composed of large numbers of peptides, oligonucleotides, or organic compounds as a mixture. These libraries are relatively easy to prepare by traditional automated synthesis methods, PCR, cloning, or synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of modulators through use of the various libraries described herein permits modification of the candidate “hit” (or “lead”) to optimize the capacity of the “hit” to modulate activity.

Candidate modulators contemplated by the invention can be designed and include soluble forms of binding partners, as well as chimeric, or fusion, proteins thereof. A “binding partner” as used herein broadly encompasses non-peptide modulators, peptide modulators (e.g., neuropeptide variants), antibodies (including monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR and/or antigen-binding sequences, which specifically recognize a polypeptide of the invention), antibody fragments, and modified compounds comprising antibody domains that are immunospecific for the expression product.

Assays that measure binding or interaction of compounds with target proteins include assays that identify compounds that inhibit unfolding or denaturation of a target protein, assays that separate compounds that bind to target proteins through affinity ultrafiltration followed by ion spray mass spectroscopy/HPLC methods or other physical and analytical methods, capillary electrophoresis assays and two-hybrid assays.

One such screening method to identify direct binding of test ligands to a target protein is described in U.S. Pat. No. 5,585,277, incorporated herein by reference. This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method which distinguishes between the folded and unfolded states of the target protein. The function of the target protein need not be known in order for this assay to be performed. Virtually any agent can be assessed by this method as a test ligand, including, but not limited to, metals, polypeptides, proteins, lipids, polysaccharides, polynucleotides and small organic molecules.

Another method for identifying ligands of a target protein is described in Wieboldt et al., Anal. Chem., 69:1683-1691 (1997), incorporated herein by reference. This technique screens combinatorial libraries of 20-30 agents at a time in solution phase for binding to the target protein. Agents that bind to the target protein are separated from other library components by simple membrane washing. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

Alternatively, such binding interactions are evaluated indirectly using the yeast two-hybrid system described in Fields et al., Nature, 340:245-246 (1989), and Fields et al., Trends in Genetics, 10:286-292 (1994), both of which are incorporated herein by reference. The two-hybrid system is a genetic assay for detecting interactions between two proteins or polypeptides. It can be used to identify proteins that bind to a known protein of interest, or to delineate domains or residues critical for an interaction. Variations on this methodology have been developed to clone genes that encode DNA binding proteins, to identify peptides that bind to a protein, and to screen for drugs. The two-hybrid system exploits the ability of a pair of interacting proteins to bring a transcription activation domain into close proximity with a DNA binding domain that binds to an upstream activation sequence (UAS) of a reporter gene, and is generally performed in yeast. The assay requires the construction of two hybrid genes encoding (1) a DNA-binding domain that is fused to a first protein and (2) an activation domain fused to a second protein. The DNA-binding domain targets the first hybrid protein to the UAS of the reporter gene; however, because most proteins lack an activation domain, this DNA-binding hybrid protein does not activate transcription of the reporter gene. The second hybrid protein, which contains the activation domain, cannot by itself activate expression of the reporter gene because it does not bind the UAS. However, when both hybrid proteins are present, the noncovalent interaction of the first and second proteins tethers the activation domain to the UAS, activating transcription of the reporter gene.

a) Antibodies to Receptors as Modulators of Binding

Standard techniques are employed to generate polyclonal or monoclonal antibodies to receptors, and to generate useful antigen-binding fragments thereof or variants thereof. Such protocols can be found, for example, in Sambrook et al., Molecular Cloning: a Laboratory Manual. Second Edition, Cold Spring Harbor, N.Y.: Cold Spring Harbor Laboratory (1989); Harlow et al. (Eds), Antibodies A Laboratory Manual; Cold Spring Harbor Laboratory; Cold Spring Harbor, N.Y. (1988). In one embodiment, recombinant polypeptides (or cells or cell membranes containing such polypeptides) are used as antigens to generate the antibodies. In another embodiment, one or more peptides having amino acid sequences corresponding to an immunogenic portion of a receptor are used as antigen. Peptides corresponding to extracellular portions of receptors, especially hydrophilic extracellular portions, are preferred. The antigen may be mixed with an adjuvant or linked to a hapten to increase antibody production. Polyclonal and monoclonal antibodies, chimeric (e.g., humanized) antibodies, fragments of antibodies, and all other forms of antibody molecules disclosed herein are referred to collectively as antibody products.

b) Polyclonal or Monoclonal Antibodies

As one exemplary protocol, a recombinant polypeptide or a synthetic fragment thereof is used to immunize a mouse for generation of monoclonal antibodies (or larger mammal, such as a rabbit, for polyclonal antibodies). To increase antigenicity, peptides are conjugated to Keyhole Lympet Hemocyanin (Pierce), according to the manufacturer's recommendations. For an initial injection, the antigen is emulsified with Freund's Complete Adjuvant and injected subcutaneously. At intervals of two to three weeks, additional aliquots of receptor antigen are emulsified with Freund's Incomplete Adjuvant and injected subcutaneously. Prior to the final booster injection, a serum sample is taken from the immunized mice and assayed by Western blot to confirm the presence of antibodies that immunoreact with a polypeptide. Serum from the immunized animals may be used as a polyclonal antisera or used to isolate polyclonal antibodies that recognize a receptor. Alternatively, the mice are sacrificed and their spleens are removed for generation of monoclonal antibodies.

One example of generating monoclonal antibodies follows: the spleens are placed in 10 ml serum-free RPMI 1640, and single-cell suspensions are formed by grinding the spleens in serum-free RPMI 1640, supplemented with 2 mM L-glutamine, 1 mM sodium pyruvate, 100 Units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a Feeder Layer. NS-1 myeloma cells, kept in log phase in RPMI with 10% (FBS(Hyclone Laboratories, Inc., Logan, Utah) for three days prior to fusion, are centrifuged and washed as well.

One example of producing hybridoma fusions follows: spleen cells from the immunized mice can be combined with NS-1 cells and centrifuged, and the supernatant is aspirated. The cell pellet is dislodged by tapping the tube, and 2 ml of 37° C. PEG 1500 (50% in 75 mM HEPES, pH 8.0) (Boehringer-Mannheim) is stirred into the pellet, followed by the addition of serum-free RPMI. Thereafter, the cells are centrifuged and resuspended in RPMI containing 15% FBS, 100 .mu.M sodium hypoxanthine, 0.4 μM aminopterin, 16 μM thymidine (HAT) (Gibco), 25 Units/ml IL-6 (Boehringer-Mannheim) and 1.5×106 thymocytes/ml and plated into 10 Corning flat-bottom 96-well tissue culture plates (Corning, Corning N.Y.).

On days 2, 4, and 6 after the fusion, 100 μl of medium is removed from the wells of the fusion plates and replaced with fresh medium. On day 8, the fusions are screened by ELISA, testing for the presence of mouse IgG that binds to a receptor polypeptide. Selected fusions are further cloned by dilution until monoclonal cultures producing anti-receptor antibodies are obtained.

(1) Receptor-Neutralizing Antibodies from Phage Display

Receptor-neutralizing antibodies are generated by phage display techniques such as those described in Aujame et al., Human Antibodies, 8(4):155-168 (1997); Hoogenboom, TIBTECH, 15:62-70 (1997); and Rader et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is screened for target-specific phage-antibodies using a labeled or immobilized target peptide or polypeptide as antigen-probe.

(2) Receptor-Neutralizing Antibodies from Transgenic Animals

Receptor-neutralizing antibodies are generated in transgenic animals, such as mice, essentially as described in Bruggemann et al., Immunol. Today 17(8):391-97 (1996) and Bruggemann et al., Curr. Opin. Biotechnol. 8:455-58 (1997). Transgenic mice carrying V-gene segments in germline configuration, and expressing the transgenes in their lymphoid tissue, are immunized with a polypeptide composition using conventional immunization protocols. Hybridomas are generated from B cells of the immunized mice using conventional protocols and screened to identify hybridomas secreting anti-receptor antibodies (e.g., as described above).

(3) High Throughput Screening (HTS) Systems for Drug Discovery

The literature is replete with examples of the use of radiolabeled ligands in HTS binding assays for drug discovery (see Williams, Med. Res. Rev. 11: 147-184 (1991); Sweetnam et al., J. Nat. Prod. 56:441-455 (1993) herein incorporated by reference in their entirety for their teaching concerning high throughput screens). It is also possible to screen for novel neuroregeneration compounds with radiolabeled ligands in HTS binding screens. Other reasons that recombinant receptors are preferred for HTS binding assays include better specificity (higher relative purity) and ability to generate large amounts of receptor material (see Hodgson, Bio/Technology 10:973-980 (1992)).

A variety of heterologous systems are available for expression of recombinant proteins and are well known to those skilled in the art. Such systems include bacteria (Strosberg et al., Trends in Pharm. Sci. 13:95-98 (1992)), yeast (Pausch, Trends in Biotech. 15:487-494 (1997)), several kinds of insect cells (Vanden Broeck, Intl. Rev. Cytol. 164:189-268 (1996)), amphibian cells (Jayawickreme et al., Curr. Opin. Biotechnol. 8:629-634 (1997)) and several mammalian cell lines (CHO, HEK293, COS, etc.; see Gerhardt et al., Eur. J. Pharmacol. 334:1-23 (1997); Wilson et al., Brit. J. Pharmacol. 125:1387-1392 (1998)). These examples do not preclude the use of other possible cell expression systems, including cell lines obtained from nematodes (WO 98/37177).

(4) Response-Based Receptor HTS systems

Inhibition of ceramide synthesis, or downstream products or genes related thereto, can result in a variety of biological responses, which are typically mediated by proteins expressed in the host cells. The proteins can be native constituents of the host cell or can be introduced through well-known recombinant technology. They can be mutants of native varieties as well. The proteins can be intact or chimeric.

Fluorescence changes can also be used to monitor ligand-induced changes in membrane potential or intracellular pH; an automated system suitable for HTS has been described for these purposes (Schroeder et al., J. Biomol. Screening 1:75-80 (1996)). Among the modulators that can be identified by these assays are natural ligand compounds; synthetic analogs and derivatives of natural ligands; antibodies, antibody fragments, and/or antibody-like compounds derived from natural antibodies or from antibody-like combinatorial libraries; and/or synthetic compounds identified by high throughput screening of libraries; and other libraries known in the art. All modulators that interact with ceramide and the synthesis pathway are useful for identifying ceramide-like polypeptides in tissue samples (e.g., for diagnostic purposes, pathological purposes, and other purposes known in the art). Agonist and antagonist modulators are useful for up-regulating and down-regulating ceramide synthesis activity, respectively, for purposes described herein.

The assays may be performed using single putative modulators; they may also be performed using a known agonist in combination with candidate antagonists (or visa versa). Detectable molecules that may be used include, but are not limited to, molecules that are detectable by spectroscopic, photochemical, biochemical, immunochemical, electrical, radioactive, and optical means, including but not limited to bioluminescence, phosphorescence, and fluorescence. These detectable molecules should be a biologically compatible molecule and should not compromise the biological function of the molecule and must not compromise the ability of the detectable molecule to be detected. Preferred detectable molecules are optically detectable molecules, including optically detectable proteins, such that they may be excited chemically, mechanically, electrically, or radioactively to emit fluorescence, phosphorescence, or bioluminescence. More preferred detectable molecules are inherently fluorescent molecules, such as fluorescent proteins, including, for example, Green Fluorescent Protein (GFP). The detectable molecule may be conjugated to the GRK protein by methods as described in Barak et al. (U.S. Pat. Nos. 5,891,646 and 6,110,693). The detectable molecule may be conjugated at the front-end, at the back-end, or in the middle.

c) Nucleic Acid Detection

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2 .mu. plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast a-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, plant cells, nematode cells, and animal cells, such as HEK-293, CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

d) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as those that interact with components of the ceramide synthase pathway, such as for example, serine palmitoyl transferase, 3-ketosphingamine reductase, dihydroceramide synthase, dihydroceramide desaturase, GlcCer synthase, sphingosine-1-phosphate lyase, sphingosine-phosphate-phosphatase, SM synthase, and sphingomyelinase, are also disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

13. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, disclosed is a kit for treating insulin resistance in a subject, comprising the compositions disclosed herein.

### D. METHODS OF USING THE COMPOSITIONS

1. Methods of Using the Compositions as Research Tools

The disclosed compositions can be used in a variety of ways as research tools. For example, the disclosed compositions can be used to study the relationship between any of the following and the molecules on which they act, as well as their relationship to each other and to other components of the ceramide synthase pathway: serine palmitoyl transferase, 3-ketosphingamine reductase, dihydroceramide synthase, dihydroceramide desaturase, GlcCer synthase, sphingosine-1-phosphate lyase, sphingosine-phosphate-phosphatase, SM synthase, and sphingomyelinase by for example acting as inhibitors of binding.

2. Methods of Gene Modification and Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in flame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination events occur at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Ceramide Synthesis is Essential for Glucocorticoid-, Saturated Fat- and Obesity-Induced Insulin Resistance

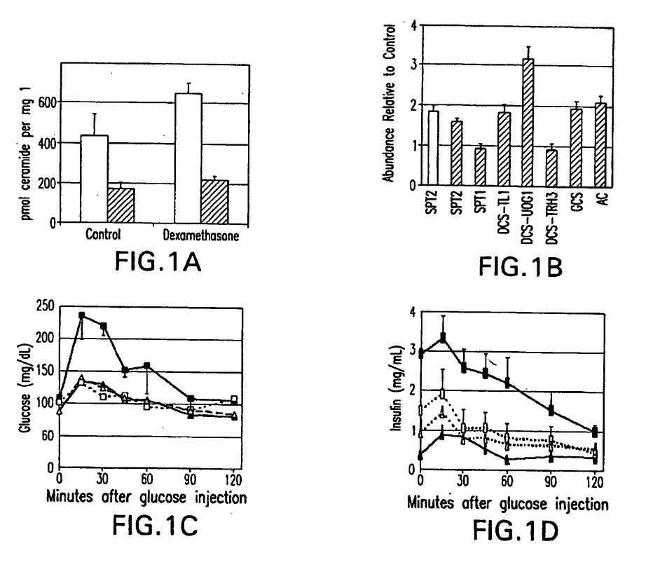

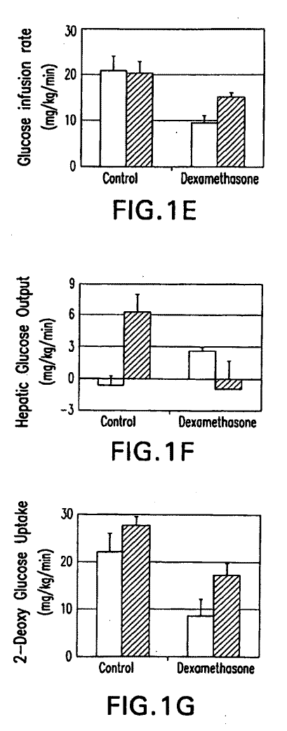

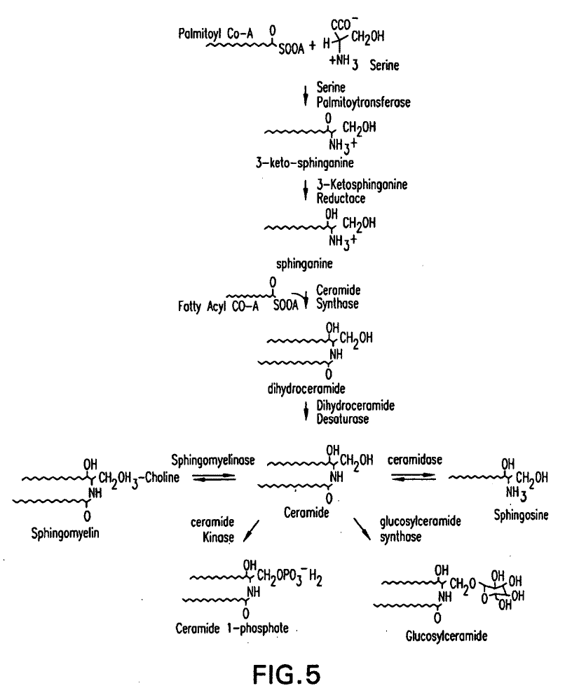

Ceramide production involves a ubiquitous biosynthetic pathway (Merrill 2002) (FIG. 5). The initial, rate-limiting reaction is the condensation of palmitoyl-CoA and serine, which is catalyzed by serine palmitoyltransferase (SPT), to produce 3-oxosphinganine. Three reactions follow resulting in the sequential production of sphinganine, dihydroceramide, and ultimately ceramide. Once generated, ceramide is the precursor of most active sphingolipids, including glucosylceramides, sphingosine, ceramide-1-phosphate, and sphingomyelin. Dexamethasone promoted ceramide accumulation in the liver (FIG. 1A) by inducing expression of serine palmitoyl transferase isoform 2 (SPT2) and dihydroceramide synthases (i.e. Translocating Chain-Associating Membrane Protein Homolog (TRH)— like protein (TL1) and Upstream of Growth and Differentiation Factor 1 (UOG1), but not TRH3 (FIG. 1B). Moreover, it promoted expression of glucosylceramide synthase and acid ceramidase, showing that glucocorticoids can also increase formation of the corresponding inhibitory sphingolipid metabolites GM3 ganglioside and sphingosine, respectively. Concomitant with the increase in ceramide accumulation, dexamethasone mildly increased (13%) fasting blood glucose (FIG. 1C) and induced a 2.8 fold increase in fasting insulin concentrations (FIG. 1D). Furthermore, dexamethasone impaired glucose disposal (FIG. 1C) and elevated insulin levels during the course of a glucose tolerance test (FIG. 1D), showing insulin resistance. Reducing ceramide levels with the SPT inhibitor myriocin (FIG. 1A) significantly negated glucocorticoid-induced glucose intolerance (FIG. 1C) and normalized circulating insulin concentrations following glucose challenge (FIG. 1D).

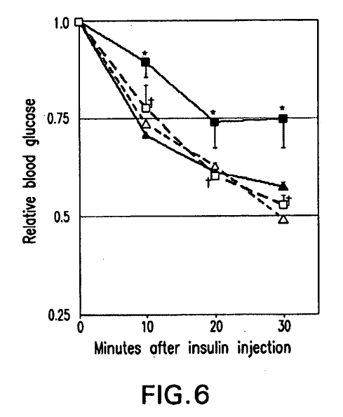

Insulin tolerance tests suggested that dexamethasone impaired glucose homeostasis by decreasing insulin sensitivity, and this effect was fully negated by myriocin (FIG. 6). To definitively evaluate effects on insulin sensitivity, as well as to distinguish whether the treatment protocols differentially affected glucose uptake in skeletal muscle or hepatic output by the liver, hyperinsulinemic-euglycemic clamps with D-[U-¹⁴C] glucose and 2-deoxy-D-[2,6-³H] glucose tracers were performed. Dexamethasone decreased the glucose infusion rate required to maintain euglycemia (FIG. 1E), prevented insulin-induced suppression of hepatic glucose output (FIG. 1F), and inhibited 2-deoxyglucose uptake into skeletal muscle. Pretreatment with myriocin partially blocked dexamethasone-induced insulin resistance in skeletal muscle, and completely prevented the glucocorticoid's effects on hepatic glucose output.

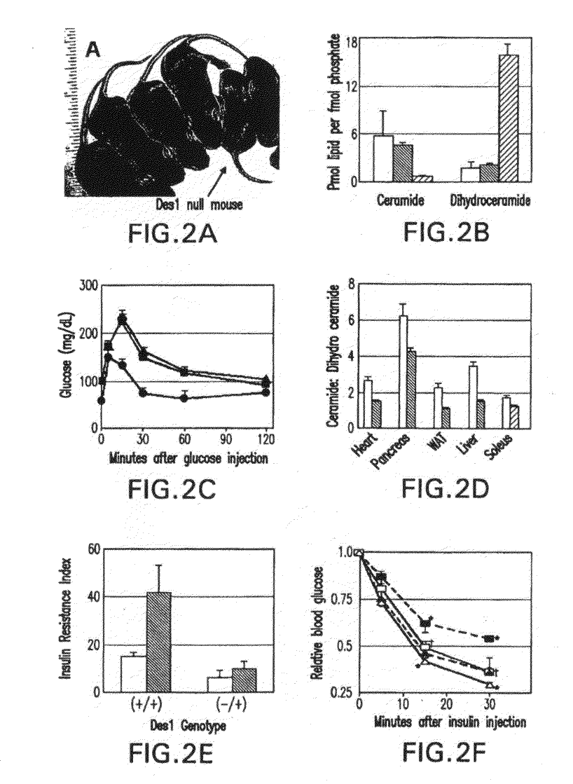

To substantiate the data obtained with myriocin, a novel genetic model was used to evaluate the role of ceramide in dexamethasone-induced insulin resistance. Genetic ablation of one or both alleles of dihydroceramide desaturase 1 (DES1) (Genbank #AF466376), which encodes the enzyme that converts metabolically inactive dihydroceramide into active ceramide in most peripheral tissues, markedly altered tissue sphingolipid levels and glucose homeostasis. One-day old homozygous null (DES1^{-/-}) pups contained much less ceramide, but dramatically more dihydroceramide (FIG. 2C), than those of wild-type (DES1^{+/+}) or haplo-insufficient (DES1^{-/+}) littermates. Moreover, heart, liver, pancreas, white adipose tissue (WAT), and soleus muscle obtained from 7-week old DES 1-animals contained undetectable ceramide levels (n=3, p<0.01), while those dissected from DES 1^{-/+} mice had a markedly reduced ratio of active ceramide to inactive dihydroceramide (FIG. 2D).

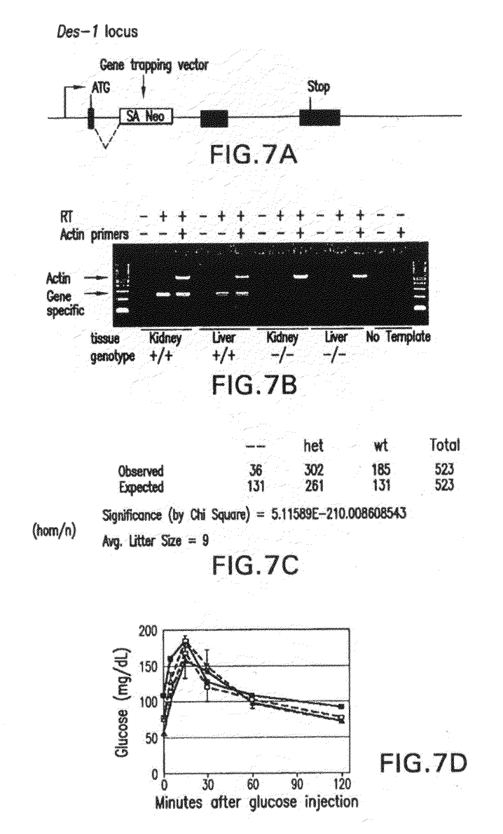

The heterozygous DES1^{-/+} animals were born at Mendelian ratios, and demonstrated no obvious health abnormalities. By contrast, the DES1^{-/-} mice revealed an incomplete penetrant lethality (FIG. 7B). Surviving animals were small in size with scaly skin and sparse hair (FIG. 2A), and ultimately failed to thrive, dying within 8 to 10 weeks of age. To ascertain the effect of DES1 ablation on glucose homeostasis, a glucose tolerance test was performed on both the DES1^{-/-} and DES1^{-/+} mice at 7 weeks of age. While the DES 1^{-/+} mice were indistinguishable from wild-type DES1^{+/+} mice, the homozygous DES 1^{-/-} animals had fasting hypoglycemia and markedly lower glucose concentrations throughout the test (FIG. 2C). By 16 weeks of age, however, the DES 1^{-/+} mice demonstrated an improvement of insulin sensitivity when compared to DES1^{+/+} littermates (FIGS. 2E and 2F) and were fully resistant to dexamethasone-induced insulin resistance (FIGS. 2E and 2F). These studies strongly support the conclusion that ceramide synthesis is requisite for glucocorticoid-induced insulin resistance.

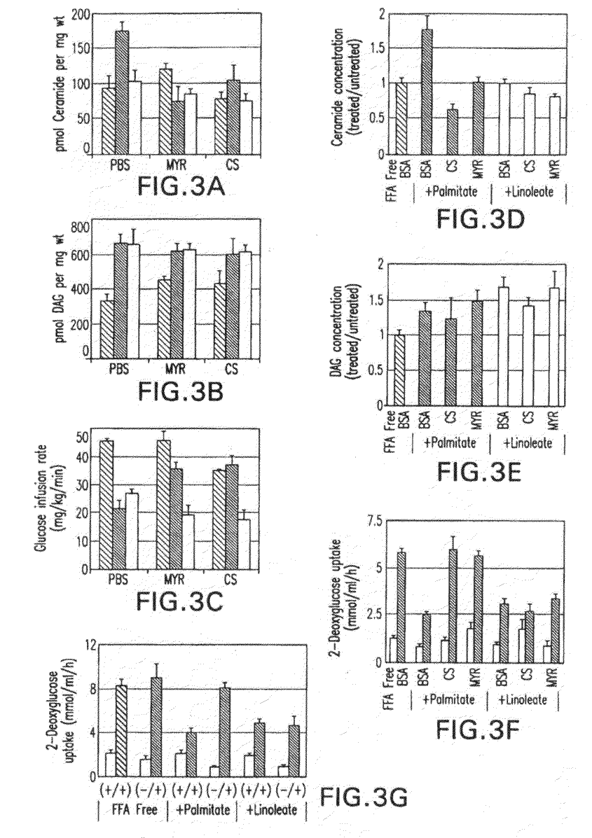

The more common mechanism by which insulin resistance likely develops in the obese is through the accumulation of fats in tissues not-suited for lipid storage. Indeed, intramyocellular lipid levels inversely correlate with insulin sensitivity, and are one of the best predictors of insulin resistance in humans. To mimic this condition in rats, a 20% lard oil-heparin infusate was delivered into the bloodstream of Sprague-Dawley rats via jugular catheters. As compared to a 2.5% glycerol infusate, lard-oil infusion increased serum FFA concentrations (Table 3), and promoted ceramide accrual in skeletal muscle (FIG. 3A). It also promoted formation of diacylglycerol (DAG), a phospholipid precursor previously implicated in lipid-induced insulin resistance (FIG. 3B). To assess muscle insulin sensitivity during the infusion, hyperinsulinemic-euglycemic clamps were performed on conscious, unrestrained rats during the terminal 90 minutes of the protocol. Lard oil infusion decreased insulin-stimulated glucose disposal to 47% of glycerol treated control subjects (FIG. 3C). However, the inclusion of myriocin or cycloserine, which blocked ceramide synthesis (FIG. 3A), restored glucose utilization in the lard-infused animals (FIG. 3C). Differences in the glucose infusion rats could not be accounted for by the slight differences in the glucose concentration that was achieved during the clamp nor to differences in steady-state insulin concentrations (Table 3). Neither myriocin nor cycloserine reduced DAG (FIG. 3B), indicating that ceramide, and not DAG, is the primary regulator of insulin resistance induced by this lard oil cocktail.

Interestingly, many studies investigating acute effects of lipids on insulin action have involved the infusion of Intralipid or Liposyn II (Boden 2001, Kim 2001, Kim 2002, Kim 2004), which are soy-based lipid cocktails enriched in the unsaturated fat linoleate, but are low in the saturated fatty acids required for synthesis of the sphingosine backbone (Chavez 2003). As shown previously, Intralipid infusion inhibited insulin-stimulated glucose disposal (FIG. 3C) without inducing ceramide accumulation (FIG. 3A). Like lard oil, however, it markedly increased circulating FFA (Table 3) and muscle DAG levels (FIG. 3B). Neither myriocin nor cycloserine prevented Intralipid-induced DAG accumulation (FIG. 3B) or insulin resistance (FIG. 3C), confirming that soy based cocktails induce insulin resistance by a ceramide-independent mechanism.

The differential sensitivity of the lard and soy oil emulsions to inhibitors of ceramide synthesis can show that different fatty acids induce insulin resistance by distinct mechanisms. The free fatty acids (FFAs) palmitate (16:0) and linoleate (18:2) were evaluated, which differ in abundance in the cocktails and in their capacity to generate ceramide, demonstrating a differential sensitivity to inhibitors of ceramide synthesis. Following completion of a six-hour incubation, both palmitate and linoleate inhibited insulin-stimulated 2-deoxyglucose (2-DOG) uptake (FIG. 3F) and induced DAG accrual (FIG. 3E) in isolated muscle strips. However, palmitate, but not linoleate, promoted ceramide accumulation (FIG. 3D). To ascertain if ceramide selectively mediated the antagonistic effects of palmitate, identical groups of muscles were treated with cycloserine (1 mM) or myriocin (10 μM). Both compounds prevented palmitate-induced ceramide accumulation without affecting DAG (FIGS. 3D and E), and the inclusion of either drug completely negated the antagonistic effects of palmitate on insulin-stimulated 2-deoxyglucose uptake (FIG. 3F). By contrast, these compounds did not alter linoleate effects on DAG accumulation (FIG. 3E) or 2-DOG uptake (FIG. 3F). Results obtained using muscles isolated from the aforementioned knockout mice showed this, as the DES1^{-/+} animals were resistant to palmitate inhibition of 2-DOG uptake, but were fully responsive to linoleate (FIG. 3G).

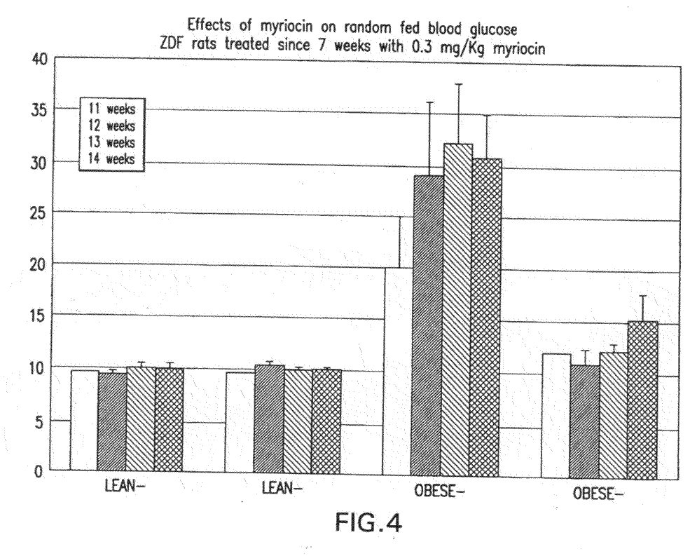

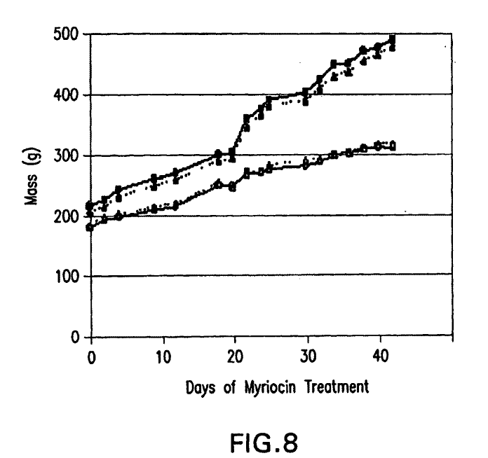

Next it was asked whether modulating ceramide levels could be an effective therapeutic strategy for combating insulin resistance and glucose intolerance in rodent models of obesity. Zucker Diabetic Fatty (ZDF) rats, which become diabetic at 10-11 weeks of age, and lean controls were given myriocin for up to 6 weeks. As in prior studies (Park 2004, Hojjati 2004), the compound was well-tolerated, as it had no effect on body mass (FIG. 8) and no apparent side effects. As predicted, animals treated with vehicle only demonstrated a progressive increase in plasma glucose (FIG. 4A) and triglycerides (FIG. 4B). Moreover, these animals failed to maintain the compensatory hyperinsulinemia seen in early stages of glucose intolerance, and the disease progressed as circulating insulin levels fell (FIG. 4C). Treatment with myriocin, starting at week 8, prevented the onset of diabetes, as evidenced by the reduced glucose and triglyceride levels in the drug-treated animals (FIG. 4A-4C). The improvement in blood glucose persisted until the animals were at least 16 weeks in age. Ceramides were elevated in liver (FIG. 4D), soleus (FIG. 4E), and serum (FIG. 4F) of the ZDF rats, and myriocin reduced levels of the sphingolipid in all these tissues. Myriocin similarly improved glucose homeostasis and lowered insulin levels in other, non-diabetic models of insulin resistance, including the Zucker fa/fa rat and DIO mice.

A collision of genetic and environmental factors has produced an epidemic expansion of obesity and insulin resistance rates during the last decade. As a result, prognosticators estimate that life expectancy, which has risen steadily over the last two centuries, may soon start to decline (Olshansky 2005). In humans, the development of insulin resistance is likely caused by a diverse number of factors (nutrients, inflammatory cytokines, and glucocorticoids) which are influenced by obesity and a sedentary lifestyle. Ceramide is a candidate molecular intermediate linking many of these metabolic stresses to the induction of insulin resistance, and the sphingolipid accumulates in insulin resistant humans. Herein it has been demonstrated that ceramide synthesis is essential for the induction of insulin resistance by at least two different pathogenic insults (i.e. glucocorticoids and saturated fatty acids). Moreover, inhibition of ceramide biosynthesis markedly improved glucose homeostasis in rodent models of obesity and diabetes. Thus, excess accumulation of ceramide and/or its metabolites likely underlies the antagonism of insulin signaling and mitochondrial function that lead to insulin resistance.

The studies presented have additional implications towards the understanding of the pathology of insulin resistance caused by lipid oversupply. Findings obtained using lipid-infusion protocols have suggested that DAG, and not ceramide, is the primary mediator of lipid-induced insulin resistance. The differences between the prior studies and those presented herein can be demonstrated by showing that ceramide synthesis is dispensable for unsaturated fat (i.e. linoleate)-induced insulin resistance, which do not drive ceramide synthesis. By contrast, however, ceramide is a requisite intermediate linking saturated fatty acids (i.e. palmitate) to the antagonism of insulin action. Thus, these findings provide definitive evidence that distinct fatty acids antagonize insulin-stimulated glucose uptake by separable mechanisms discerned by their intracellular routes of metabolism, and that prior studies relying on lipid cocktails comprised predominantly of unsaturated fatty acids may have underappreciated the roles of ceramides as antagonists of insulin action. Since both dietary and epidemiological studies show that saturated fats, which are more poorly oxidized than unsaturated ones (Gaster 2005) in the induction of human insulin resistance (Rivellese 2003), modulators of ceramide may prove useful for combating insulin resistance caused from the oversupply of this class of lipid nutrients.

In summary, using an array of pharmacological and genetic tools to modulate ceramide levels in rodents, the following has been demonstrated: first, ceramide is common molecular intermediate linking both glucocorticoids and saturated fatty acids (i.e. palmitate) to the induction of insulin resistance; second, different fatty acid classes antagonize insulin-stimulated glucose uptake by separable mechanisms discerned by their dependence upon ceramide; and third, manipulating ceramide levels in obese rodents ameliorates insulin resistance and blocks the onset of diabetes.

a) METHODS

(1) Materials

Myriocin and L-cycloserine were obtained from sigma chemicals (St. Louis, Mo.). Humalin human insulin was from Eli Lilly (Indianapolis, Ind.).

(2) Animals

All animals were received from Charles River Laboratories (Wilmington, Mass.). Upon arrival, rats were housed four to a cage in a temperature-controlled animal room maintained on a 12:12-h light-dark cycle. The rats were fed ad libitum National Institutes of Health standard chow and water. For lipid infusion and hyperinsulinemic euglycemic clamps animals were anesthetized with ketamine (65 mg/Kg) and xylazine (10 mg/Kg) and polyethylene catheters (Clay Adams Intramedic, PE-50, Bectin-Dickinson, Sparks, Md.) were asceptically placed in the left carotid artery (advanced to the aortic arch) or the right jugular vein. Catheters were filled with 3% heparinized saline to maintain patent, and exteriorized in the intrascapular region. Buprenorphine (0.03 mg/Kg, sc) was administered post surgically for pain control and animals were allowed to recover (5-7 days) to preoperative weight prior to experiments.

(3) Measurement of Plasma Substrates and Insulin

Tail vein blood (3 μl) was sampled for glucose determination (Hitachi 911 clinical chemistry analyzer, Roche, Indianapolis, Ind. or Glucometer Elite, Bayer Corp., Tarrytown, N.Y.)-30 minutes, immediately before and 15, 30, 45, 60, 90, and 120 min after glucose administration. Additionally, serum was collected and analyzed for insulin by Elisa (Linco Diagnostics, Springfield, Mo.).

(4) Des1 Knockout Mice

Mouse Embryonic Stem (ES) cells carrying a mutation in the Des-1 gene (accession number NM_{—}007853) were obtained from OmniBank, a library of gene-trapped ES cell clones identified by a corresponding OmniBank Sequence Tag (OST) (Zambrowicz et al, 1998, Zambrowicz et al, 2003). The ES cell clone corresponding to OST 368559, matching the mouse Des-1 sequence, was thawed and expanded. Inverse genomic PCR (Silver, 1989) analysis of DNA from this clone confirmed the insertion of the gene trapping retroviral vector in the first intron of the Des-1 gene on chromosome 1, downstream of the translation initiation codon thus disrupting production of wild-type transcripts (FIG. 5A). OST 368559 cells were used to generate mice heterozygous for the Des-1 mutation using standard methods (Joyner, 2000). Genotyping of mice was carried out using a multiplex PCR strategy on genomic DNA as described (Schrick et al, 2006).

Generation of Des-1 mutant ES cells and mice: The generation of the OmniBank gene trap library has been described (Zambrowicz et al 1998, Zambrowicz et al 2003). Des-1 mutant mice were generated by microinjection of ES cells clones into host blastocysts using standard methods (Joyner, 2000). The precise genomic insertion site of the retroviral gene trapping vector in the Des-1 gene was determined by inverse genomic PCR as described (Silver, 1989).

Mouse Genotyping: Oligonucleotide primers (LTR2, forward: 5′-AAATGGCGTTACTTAAGCTAGCTTGC-3′ (SEQ ID NO: 3), and gene specific primer, reverse: 5′-AGCTAGTCACTCTGATTTGCGAAAC-3′, SEQ ID NO: 4) were employed in a multiplex reaction to amplify mutant Des-1 alleles as described (Schrick et al, 2006).

Des-1 RT-PCR: RNA was extracted from liver and kidney using a bead homogenizer and RNAzol (Ambion) according to the manufacturer's instructions. Reverse transcription was performed with SuperScript II (Invitrogen) and random hexamer primers, according to the manufacturer's instructions. PCR amplification (95° C., 30 seconds, 59° C., 45 seconds, 70° C., 60 seconds) was performed for 30 cycles using primers complementary to exons 1 and 2 of the Des-1 gene, flanking the insertion site of the vector (forward: 5′-GGGTCCACAGGCCGCAGCCATGGGTAGC-3′ (SEQ ID NO: 5) and reverse: 5′-GCCAAAGACATAGGACCAAAATATGACCCA-3′, SEQ ID NO: 6). Control primers to the mouse β-Actin gene (accession number NM_{—}007393) were:5′GGCTGGCCGGGACCTGACGGACTACCTCAT-3′ (SEQ ID NO: 7) and 5′-GCCTAGA AGCACTTGCGGTGCACGATGGAG-3′ (SEQ ID NO: 8) Des-1 RT-PCR products were verified by sequencing.

(5) Lipid Infusion

Male Sprague-Dawley rats (˜250 grams) were randomly divided into nine different groups. Glycerol, 20% lard oil, or 20% soy oil emulsions were prepared as previously described (Stein 1997) and infused at a constant rate of 5 mL/Kg/hour. To activate lipoprotein lipase, heparin (6 units/hour) was added to the triglyceride emulsions. Lipids were infused in animals treated with control injections (normal saline), myriocin (100 μg/Kg IP) or cycloserine (50 mg/Kg IP). Drugs were given 12 hours prior to lipid infusion and equivalent doses were given intravenously at the time of infusion. Hyperinsulinemic euglcemic clamps were initiated after 4.5 hours of infusion and lipids were coinfused during the duration of the procedure. Following lipid infusion animals were deeply anesthetized with sodium pentobarbital and soleus muscles were rapidly dissected out and rapidly frozen in liquid nitrogen.

(6) Isolated Muscles

Palmitate or linoleate was first dissolved in ethanol (200 mM), and free fatty acids or ethanol was conjugated to BSA by diluting 1:25 in Krebs-Henseleit buffer (KHB) supplemented with 20% BSA, and heating (55° C., 30 minutes with occasional vortexing). The final incubation media was prepared by diluting the conjugated BSA solution 1:8 in freshly oxygenated KHB. Male Sprague Dawley rats (150-200 grams) were deeply anesthetized with sodium pentobarbital (110 mg/Kg IP), and soleus muscles were isolated, laterally bisected and then transferred to 25-ml Erlenmeyer flasks containing 2 ml of KHB supplemented with 2.5% bovine serum albumin (BSA), 8 mM glucose, and 1 mM HEPES (pH 7.2). Muscles were maintained in a shaking water bath at 29° C. while being continuously gassed with 95% O₂/5% CO₂. Following this incubation, muscles were rapidly frozen in liquid nitrogen or stimulated with insulin (300 μU) for 60 minutes and the incorporation of ³H-2-deoxy-D-glucose was assessed during the final 20 minutes of the incubation using the method of Brozinick and Birnbaum (Brozinick 1998). Free fatty acids were present throughout the glucose uptake assay.

(7) Zucker Diabetic Fatty Rats

Male ZDF rats were obtained from Charles River (Genetic Models, Inc, Indianapolis, Ind., USA) at 6 weeks of age. After a 2-week acclimation period, rats were pre-bled and assigned to four groups (5 animals per group; vehicle, myriocin at 0.25 or 0.5 mg/kg/day or L-cycloserine at 25 or 50 mg/kg/day) based on starting plasma glucose levels and body weight (day −1). Rats were administered compound daily by oral gavage between 8:30 and 9:30 AM for seven days. The dosing vehicle was 1% w/v carboxymethylcellulose, 0.25% Tween-80. Blood samples were obtained 1 hour postdose at the indicated days from the tail vein of conscious animals by gentle massage following tail-snip. Blood was collected in EDTA tubes and kept chilled on ice. Following centrifugation of blood samples, plasma was used for measurements of glucose, adiponectin, and triglyceride levels. Statistical significance was determined by one-way ANOVA. When statistical significance was detected with this method, group differences were determined by Neuman-Keuls post-hoc analyses.

(8) Hyperinsulinemic Euglycemic Clamps

Clamps were performed in conscious unrestrained animals using swivels and tethers (Instech, Plymouth Meeting, Pa.) to allow uninterrupted movement of the animal without disruption of infusion lines. Hyperinsulinemia was initiated by intravenous infusion of insulin (10 mU/kg/min at a flow rate of 4 μL/min). Blood was sampled from arterial lines in 7 minute intervals and analyzed with a Beckman Glucose analyzer II (Beckman Coulter, Fullerton, Calif.). Euglycemia was maintained by variable infusion of 20% dextrose. Steady state was achieved approximately 90 minutes after initiating hyperinsulinemia and maintained for 30 minutes. Glucose infusion rates were calculated as the average glucose infusion rate during the 30 minute steady state. Additional blood samples were taken before initiating hyperinsulinemia and at the end of the clamp for analysis of insulin and free fatty acids.

TABLE 3
Dexamethasone Clamps

| | | Before Clamp | | | During Clamp | |
|---|---|---|---|---|---|---|
| Treatment | | Glucose | Insulin | | Glucose | Insulin |
| Dexamethasone | Myriocin | (mg/dL) | (ng/mL) | FFA (μM) | (mg/dL) | (ng/mL) |
| Control | Saline | 109.4 ± 8.7 | 1.14 ± 0.31 | 240 ± 27 | 128.2 ± 3.2 | 5.7 ± 1.0 |
| | Myriocin | 123.6 ± 7.7 | 2.05 ± 0.25 | 258 ± 29 | 130.5 ± 2.9 | 6.4 ± 0.5 |
| Dexamethasone | Saline | 139.0 ± 3.1* | 4.36 ± 2.34 | 211 ± 38 | 131.1 ± 5.1 | 6.7 ± 1.3 |
| | Myriocin | 137.0 ± 2.1 | 2.34 ± 0.49 | 176 ± 34 | 129.3 ± 3.7 | 5.4 ± 1.4 |

Table 3. Plasma parameters were assessed during hyperinsulinemic euglycemic clamps of dexamethasone treated Sprague dawley rats. Plasma was sampled from indwelling arterial lines before the infusion of insulin, or during a 60 minute euglycemic steady state. Glucose was analyzed using a Beckman Glucose analyzer II (Beckman Coulter, Fullerton, CA) approximately every 7 minutes throughtout the steady state period. Insulin was analyzed by enzyme linked immunosorbent assay from 2 steady state samples. Free Fatty Acid (FFA) was measured using a colorometric assay (half-micro test, Roche). Data are presented as the mean ± standard error (n = 4).

TABLE 4
Lipid Infusion Clamps

| | | Glucose (mg/dL) | | | Insulin (ng/mL) | | |
|---|---|---|---|---|---|---|---|
| Treatment | | Before | Before | During | Before | Before | During |
| Lipid | Drug | Infusion | Clamp | Clamp | Infusion | Clamp | Clamp |
| Glycerol | PBS | 96.0 ± 7.1 | 109.8 ± 2.3 | 111.7 ± 2.1 | 0.471 ± 0.09 | 0.662 ± 0.03 | 11.54 ± 1.27 |
| | Myriocin | 103.0 ± 10.6 | 117.7 ± 9.1 | 111.2 ± 6.6 | 0.436 ± 0.03 | 1.90 ± 0.71 | 8.02 ± 2.52 |
| | L-cycloserine | 104.0 ± 13.1 | 113.0 ± 3.2 | 111.0 ± 3.0 | 0.659 ± 0.11 | 9.19 ± 0.20 | 12.08 ± 0.03 |
| Lard oil | PBS | 95.3 ± 5.5 | 96.7 ± 2.6 | 105.4 ± 1.3 | 0.515 ± 0.11 | 3.78 ± 0.64 | 14.78 ± 0.99 |
| | Myriocin | 101.0 ± 11.4 | 123.3 ± 3.8 | 108.4 ± 1.0 | 0.407 ± 0.47 | 1.90 ± 0.50 | 17.02 ± 3.79 |
| | L-cycloserine | 100.6 ± 6.4 | 101.6 ± 7.1 | 106.6 ± 2.6 | 0.793 ± 0.15 | 2.18 ± 0.76 | 16.27 ± 2.69 |
| Soy oil | PBS | 100.6 ± 6.4 | 101.1 ± 3.1 | 106.6 ± 0.7 | 0.630 ± 0.03 | 3.13 ± 1.1 | 9.21 ± 1.29 |
| | Myriocin | 109.3 ± 10.4 | 105.0 ± 4.6 | 108.3 ± 0.4 | 0.305 ± 0.08 | 2.46 ± 1.1 | 11.55 ± 2.91 |
| | L-cycloserine | 102.3 ± 7.1 | 124.0 ± 10.8 | 106.2 ± 0.44 | 1.12 ± 0.17 | 3.13 ± 1.0 | 14.38 ± 0.67 |

| | | FFA (mM) | | |
|---|---|---|---|---|
| Treatment | | Before | Before | During |
| Lipid | Drug | Infusion | Clamp | Clamp |
| Glycerol | PBS | 0.485 ± 0.07 | 0.470 ± 0.09 | 0.414 ± 0.11 |
| | Myriocin | 1.22 ± 0.25 | 0.455 ± 0.13 | 0.872 ± 0.75 |
| | L-cycloserine | 0.562 ± 0.10 | 0.489 ± 0.10 | 0.501 ± 0.11 |
| Lard oil | PBS | 0.556 ± 0.05 | 1.35 ± 0.37 | 1.56 ± 0.19 |
| | Myriocin | 0.912 ± 0.30 | 1.52 ± 0.49 | 2.427 ± 0.30 |
| | L-cycloserine | 0.672 ± 0.14 | 1.53 ± 0.19 | 2.12 ± 0.89 |
| Soy oil | PBS | 0.619 ± 0.11 | 1.21 ± 0.07 | 1.64 ± 0.17 |
| | Myriocin | 1.01 ± 0.10 | 1.58 ± 0.57 | 1.80 ± 0.44 |
| | L-cycloserine | 1.02 ± 0.32 | 1.51 ± 0.23 | 2.10 ± 0.77 |

Table 4. Plasma parameters were assessed during hyperinsulinemic euglycemic clamps of lipid infused Sprague-Dawley rats. Plasma was sampled from indwelling arterial lines before the infusion of lipid, before coinfusion of insulin, or during a 30 minute euglycemic steady state. Glucose was analyzed using a Beckman Glucose analyzer II (Beckman Coulter, Fullerton, CA) approximately every 5 minutes throughtout the steady state period. Insulin was analyzed by enzyme linked immunosorbent assay from terminal plasma samples. Free Fatty Acid (FFA) was measured using a colorometric assay (Roche). Data are presented as the mean ± standard error (n = 4).

### 2. Example 2

### DES-1 Knock Out Animals

Ceramide accumulation within muscle and liver induces insulin resistance by directly antagonizing insulin action. Ceramide can also have an extrahepatic or extramuscular role, and its synthesis in other tissues (i.e. adipose tissue, vasculature, etc.) is most relevant in the induction of insulin resistance. A conditional knockout mouse has been developed which can be used to mouse strains lacking DES1 in skeletal muscle, adipose tissue, and the liver. The creation of these mouse strains allows for the evaluation of the consequence of complete ceramide ablation in insulin-responsive tissues in the absence of some of the health abnormalities present in the whole animal knockout. Moreover, it allows for the identification of tissues that are particularly sensitivity to ceramide accrual. The consequence of depleting DES 1 in muscle, adipose tissue, and/or liver on glucocorticoid-induced insulin resistance can be measured by measuring the following: insulin-stimulated glucose disposal, both in the whole animal and in these tissues; suppression of hepatic glucose output; and, tissue and serum ceramide levels. Collectively these experiments give insight into the primary target of ceramide's deleterious effects.

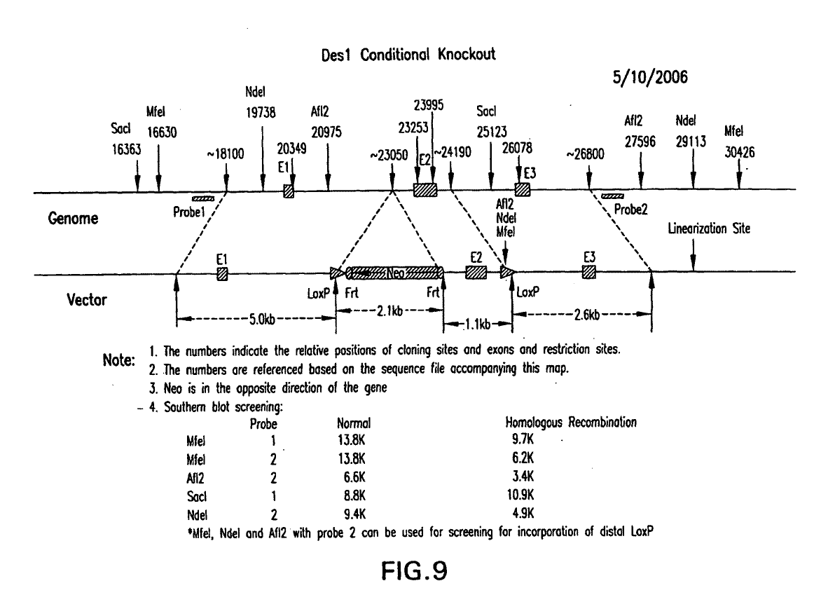

Generation of a conditional DES1 knockout mouse (FIG. 9). DES1 (NP_{—}031879) contains three exons, with the majority of the translated region encoded by exon 2. Vega Biolabs (Philadelphia, Pa.) can generate the conditional knockout vector, which can be achieved by flanking exon 2 with loxP sites. This group has isolated and purified the BAC clone and designed and constructed a targeting vector containing an Flp-flanked neomycin resistance marker and a loxP-flanked exon 2. The knockout mouse is be prepared using the University of Utah transgenic and knockout core facility, which is electroporating and screening embryonic stem cells, excising the selectable Neo cassette, performing blastocyst injections, and breeding for germline transmission and homozygosity.

Generation of muscle, adipose tissue, and liver-specific knockouts: Mice are backcrossed onto the C57/B16 background prior to breeding them with the transgenic animals expressing cre-recombinase. For preparation of liver, adipose tissue, or muscle-specific knockouts, mice are crossed with albumin-cre, AP2-Cre, or muscle creatine kinase-cre transgenic mice, respectively. Genotyping of mice containing Cre is done by PCR of genomic DNA obtained by tail snip using established primers (5′-ATGTCCAATTTACTGACCG-3′ (SEQ ID NO: 9) and 5′-CGCCGCATAACCAGTGAAAC-3′, SEQ ID NO: 10). Genotyping of DES1 is done by PCR using primers recommended by Vega Biolabs.

### 3. Example 3

### TNFα Alters the Expression of Genes Controlling Ceramide Synthesis and Degradation

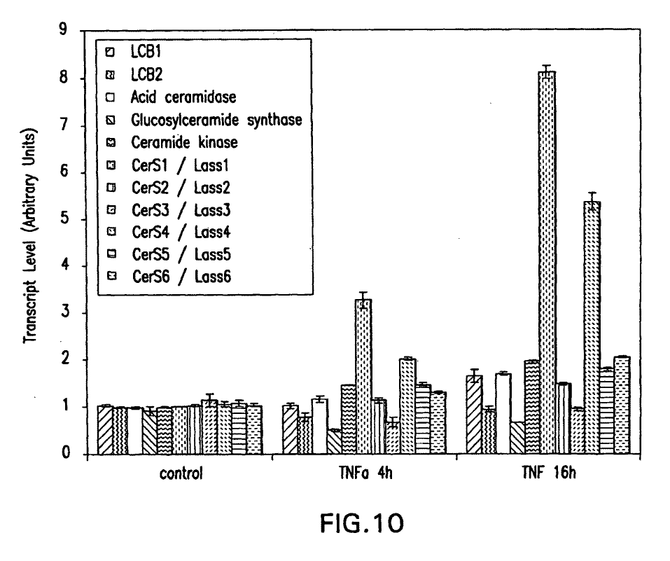

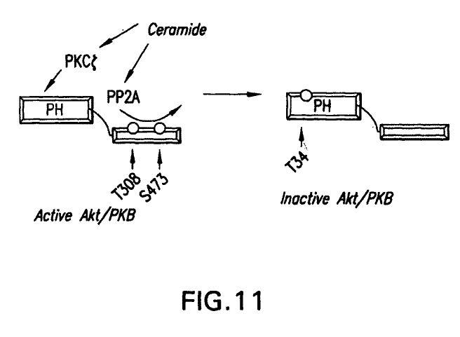

The possibility that TNFαα-stimulation of lipolysis can account for the increase in ceramide by stimulating the formation of intracellular palmitoyl-CoA, which could serve as a precursor for ceramide biosynthesis, was considered. However, blocking lipolysis with a MAPK inhibitor failed to prevent this TNFα effect. Moreover, performing these experiments in the absence of glucose, which prevents TNFα-stimulated lipolysis did not prevent its induction of ceramide. Instead, it appears that TNFα regulates the expression or activity of enzymes that control intracellular ceramide levels. Using RT-PCR, changes in mRNA levels of genes which control ceramide synthesis or degradation were evaluated. It was shown that TNFα increased expression of the two subunits of serine palmitoyltransferase, LCB1 and LCB2, and markedly reduced expression of glucosylceramide synthase (FIG. 10). The analysis was extended to consider several ceramide synthase (CerS/LASS) isoforms, which selectively catalyze the incorporation of distinct fatty acids into the sphinganine backbone. TNFα-induced an impressive 8 and 5-fold increase in CerS1 and CerS4, respectively. These enzymes preferentially catalyze the production of ceramide species containing the stearate fatty acid. These observations show that specific ceramide subspecies containing this fatty acid side chain can be preferentially involved in TNFα-induced insulin resistance.

### F. SEQUENCES

SEQ ID NO: 1
MGSRVSREDFEWVYTDQPHADRRREILAKYPEIKSLMKPDPNLIWIIIMM
VLTQLGAFYIVKDLDWKWVIFGAYAFGSCINHSMTLAIHEIAHNAAFGNC
KAMWNRWFGMYANLPIGIPYSISFKRYHMDHHRYLGADGVDVDIPTDFEG
WFFCTAFRKFIWVILQPLFYAFRPLFINPKPITYLEVINTVAQVTFDILI
YYFLGIKSLVYMLAASLLGLGLHPISGHFIAEHYMFLKGHETYSYYGPLN
LLTFNVGYHNEHHDFPNIPGKSLPLVRKIAAEYYDNLPHYNSWIKVLYDF
VMDDTISPYSRMKRHQKGEMVLE
SEQ ID NO: 2
cacagccggc cgacaccaca ccagccgggg agccgccgcc
gccgccgcca cctctgagca gccggctggg agcgagagcc
gacagctagt ctgcaagcca ccgctgtcgc catggggagc
cgcgtctcgc gggaagactt cgagtgggtc tacaccgacc
agccgcacgc cgaccggcgc cgggagatcc tggcaaagta
tccagagata aagtccttga tgaaacctga tcccaatttg
atatggatta taattatgat ggttctcacc cagttgggtg
cattttacat agtaaaagac ttggactgga aatgggtcat
atttggggcc tatgcgtttg gcagttgcat taaccactca
atgactctgg ctattcatga gattgcccac aatgctgcct
ttggcaactg caaagcaatg tggaatcgct ggtttggaat
gtttgctaat cttcctattg ggattccata ttcaatttcc
tttaagaggt atcacatgga tcatcatcgg taccttggag
ctgatggcgt cgatgtagat attcctaccg attttgaggg
ctggttcttc tgtaccgctt tcagaaagtt tatatgggtt
attcttcagc ctctctttta tgcctttcga cctctgttca
tcaaccccaa accaattacg tatctggaag ttatcaatac
cgtggcacag gtcacttttg acattttaat ttattacttt
ttgggaatta aatccttagt ctacatgttg gcagcatctt
tacttggcct gggtttgcac ccaatttctg gacattttat
agctgagcat tacatgttct taaagggtca tgaaacttac
tcatattatg ggcctctgaa tttacttacc ttcaatgtgg
gttatcataa tgaacatcat gatttcccca acattcctgg
aaaaagtctt ccactggtga ggaaaatagc agctgaatac
tatgacaacc tccctcacta caattcctgg ataaaagtac
tgtatgattt tgtgatggat gatacaataa gtccctactc
aagaatgaag aggcaccaaa aaggagagat ggtgctggag
taaatatcat tagtgccaaa gggattcttc tccaaaactt
tagatgataa aatggaattt ttgcattatt aaacttgaga
ccagtgatgc tcagaagctc ccctggcaca atttcagagt
aagagctcgg tgataccaag aagtgaatct ggcttttaaa
cagtcagcct gactctgtac tgctcagttt cactcacagg
aaacttgtga cttgtgtatt atcgtcattg aggatgtttc
actcatgtct gtcattttat aagcatatca tttaaaaagc
ttctaaaaag ctatttcgcc aggcacggtg gctcatgcct
ataatcccag cactttggga ggccaaggtg ggtggatcac
ctgaggtcag gagttcgaga ccagcctggc caacacggtg
aaaccccatc tctactaaaa atgcaaaaat tagccgggcg
tggcggcaca tgcctgtaat cccagctaca tgggaggctg
aggtgggaga attgcttgaa cccaggaggc ggaggcagag
gctgcagtga cccaagattg tgccactgca ctccaccctg
ggcaacagag caagacccca tctcaaaaat aaataaatat
atataaaaaa taaaaagcta tttctagttt atttcactat
aaagttttgc tttattaaaa agctaataaa cagctattaa
tcacagtgta ttagtatttg ttacattttt gtatttcact
atctttatac tatataatat ggtaacttgg gtaccggggg
aactttaaaa tttcatctca aaaataattt ttaaaaagcc
tgaggtatga tatagcataa aagattgaga tgaaaatata
tttccctgta agctgaatta ctcatttaaa aattttaact
tctatatggg acccgaatta gacactgctg aatcctgtac
agccttactc ataaataaag tacttactga atttccacca
ttcaaaaaaa aaaaaaaa
SEQ ID NO: 3
AAATGGCGTTACTTAAGCTAGCTTGC
SEQ ID NO: 4
AGCTAGTCACTCTGATTTGCGAAAC
SEQ ID NO: 5
GGGTCCACAGGCCGCAGCCATGGGTAGC
SEQ ID NO: 6
GCCAAAGACATAGGACCAAAATATGACCCA
SEQ ID NO: 7
GGCTGGCCGGGACCTGACGGACTACCTCAT
SEQ ID NO: 8
GCGTAGA AGCACTTGCGGTGCACGATGGAG
SEQ ID NO: 9
ATGTCCAATTTACTGACCG
SEQ ID NO: 10
CGCGGCATAACCAGTGAAAC

### G. REFERENCES

1. G. M. Reaven, Annu Rev Nutr 25, 391 (2005).
2. A. Hermanowski-Vosatka et al., J Exp Med 202, 517 (Aug. 15, 2005).
3. G. S. Hotamisligil, Int J Obes Relat Metab Disord 27 Suppl 3, S53 (December, 2003).
4. C. Schmitz-Peiffer, Cell Signal 12, 583 (2000).
5. J. D. McGarry, Diabetes 51, 7 (2002).
6. S. A. Summers, D. H. Nelson, Diabetes 54, 591 (March, 2005).
7. C.-N. Wang, L. O'Brien, D. N. Brindley, Diabetes 47, 24 (1998).
8. S. A. Summers, L. A. Garza, H. Zhou, M. J. Bimbaum, Mol Cell Biol 18, 5457 (1998).
9. J. A. Chavez, W. L. Holland, J. Bar, K. Sandhoff, S. A. Summers, J Biol Chem 280, 20148 (May 20, 2005).
10. J. A. Chavez et al., J Biol Chem 13, 10297 (2003).
11. D. J. Powell, E. Hajduch, G. Kular, H. S. Hundal, Mol Cell Biol 23, 7794 (November, 2003).
12. D. J. Powell, S. Turban, A. Gray, E. Hajduch, H. S. Hundal, Biochem J 382, 619 (Sep. 1, 2004).
13. P. P. Ruvolo, Pharmacol Res 47, 383 (May, 2003).
14. M. Yuan et al., Science 293, 1673 (Aug. 31, 2001).
15. J. Hirosumi et al., Nature 420, 333 (Nov. 21, 2002).
16. T. I. Gudz, K. Y. Tsemg, C. L. Hoppel, J Biol Chem 272, 24154 (Sep. 26, 1997).
17. L. J. Siskind, R. N. Kolesnick, M. Colombini, J Biol Chem 277, 26796 (Jul. 26, 2002).
18. N. Houstis, E. D. Rosen, E. S. Lander, Nature 440, 944 (Apr. 13, 2006).
19. B. B. Lowell, G. I. Shulman, Science 307, 384 (Jan. 21, 2005).
20. D. H. Nelson, D. K. Murray, Biochem Biophys Res Commun 138, 463 (Jul. 16, 1986).
21. S. Tagami et al., J Biol Chem 277, 3085 (Feb. 1, 2002).
22. C. Yu et al., J Biol Chem 277, 50230 (2002).
23. S. I. Itani, N. B. Ruderman, F. Schmieder, G. Boden, Diabetes 51, 2005 (2002).
24. H. Masuzaki et al., Science 294, 2166 (Dec. 7, 2001).
25. H. Masuzaki et al., J Clin Invest 112, 83 (July, 2003).
26. Y. Kotelevtsev et al., J Clin Invest 103, 683 (March, 1999).
27. Y. Kotelevtsev et al., Proc Natl Acad Sci USA 94, 14924 (Dec. 23, 1997).
28. A. H. Merrill, Jr., J Biol Chem 277, 25843 (2002).
29. G. Boden, G. I. Shulman, Eur J Clin Invest 32 Suppl 3, 14 (2002).
30. J. K. Kim et al., J Clin Invest 114, 823 (September, 2004).
31. J. K. Kim et al., J Clin Invest 108, 437 (2001).
32. Y. B. Kim, G. I. Shulman, B. B. Kahn, J Biol Chem 277, 32915 (Sep. 6, 2002).
33. C. Yu et al., J Biol Chem 277, 50230 (2002).
34. J. A. Chavez, S. A. Summers, Archives of Biochemistry and Biophysics 419, 101 (2003).
35. T. S. Park et al., Circulation 110, 3465 (Nov. 30, 2004).
36. M. Hojati et al., J Biol Chem (Dec. 6, 2004).
37. S. J. Olshansky et al., N Engl J Med 352, 1138 (Mar. 17, 2005).
38. J. M. Adams, 2nd et al., Diabetes 53, 25 (January, 2004).
39. M. Gaster, A. C. Rustan, H. Beck-Nielsen, Diabetes 54, 648 (March, 2005).
40. A. A. Rivellese, S. Lilli, Biomed Pharmacother 57, 84 (March, 2003).
41. D. T. Stein et al., J Clin Invest 100, 398 (Jul. 15, 1997).
42. J. T. Brozinick, Jr., M. J. Birnbaum, J Biol Chem 273, 14679 (1998).
43. Zambrowicz B P, Friedrich G A, Buxton E C, Lilleberg S L, Person C, Sands A T. (1998) Disruption and sequence identification of 2,000 genes in mouse embryonic stem cells. Nature 392(6676):608-11.
44. Zambrowicz B P, Abuin A, Ramirez-Solis R, Richter U J, Piggott J, BeltrandelRio H, Buxton E C, Edwards J, Finch R A, Friddle C J, Gupta A, Hansen G, Hu Y, Huang W, Jaing C, Key B W Jr, Kipp P, Kohlhauff B, Ma Z Q, Markesich D, Payne R, Potter D G, Qian N, Shaw J, Schrick J, Shi Z Z, Sparks M J, Van Sligtenhorst I, Vogel P, Walke W, Xu N, Zhu Q, Person C, Sands A T. (2003) Wnk1 kinase deficiency lowers blood pressure in mice: a gene-trap screen to identify potential targets for therapeutic intervention. Proc. Natl. Acad. Sci. USA. 100(24):14109-14.
45. Silver, J. & Keerikatte, V. (1989) J Virol 63, 1924-1928.
46. Schrick J. J., Vogel P., Abuin A., Hampton B., Rice D. S. (2006) ADP-ribosylation factor-like 3 is involved in kidney and photoreceptor development. Am J. Pathol. 168(4): 1288-98.
47. Joyner, A. L. (2000) Gene Targeting: A Practical Approach (Oxford University Press, Oxford, England).

What is claimed is:

1. A method of modulating insulin resistance in a cell, comprising identifying a cell in need of modulated insulin resistance, and administering to the cell a composition which inhibits ceramide synthesis, thereby modulating insulin resistance in a cell.

2. The method of claim 1, wherein the cell is in vitro.

3. The method of claim 2, wherein the cell is in vivo.

4. The method of claim 3, wherein the cell is in a subject.

5. The method of claim 4, wherein the subject has metabolic syndrome.

6. The method of claim 4, wherein the subject is obese.

7. The method of claim 4, wherein the subject has diabetes.

8. The method of claim 4, wherein the subject has Cushing's disease.

9. The method of claim 4, wherein the subject has inflammation.

10. The method of claim 4, wherein the subject has Gaucher disease.

11. The method of claim 1, wherein ceramide synthesis is inhibited by altering the expression of TNFα.

12. The method of claim 1, wherein the composition is fenretinide.

13. The method of claim 1, wherein the composition is resveratrol.

14. The method of claim 1, wherein the composition inhibits any one or more of the following: serine palmitoyl transferase, 3-ketosphingamine reductase, dihydroceramide synthase, dihydroceramide desaturase, GlcCer synthase, sphingosine-1-phosphate lyase, sphingosine-phosphate-phosphatase, SM synthase, and sphingomyelinase.

15. The method of claim 14, wherein the compound is dihydroceramide desaturase (DES).

16. A method of screening for a test compound that modulates ceramide synthesis comprising:
a. contacting a cell that produces ceramide with a test compound; and
b. detecting altered levels of ceramide synthesis; wherein altered levels of ceramide synthesis indicate a compound that modulates ceramide synthesis.

17. The method of claim 16 wherein the cell is contacted with a plurality of test compounds in a high throughput assay system.

18. The method of claim 17, wherein the high throughput assay system comprises an immobilized array of test compounds.

19. A compound identified by the method of claim 16.

20. A method of screening for a test compound that modulates ceramide synthesis comprising:
a. contacting a transgenic animal that is deficient in one or more of the following proteins: serine palmitoyl transferase, 3-ketosphingamine reductase, dihydroceramide synthase, dihydroceramide desaturase, GlcCer synthase, sphingosine-1-phosphate lyase, sphingosine-phosphate-phosphatase, SM synthase, and sphingomyelinase with a test compound; and
b. detecting a difference in ceramide synthesis in the transgenic animal; wherein a difference in ceramide synthesis indicates a test compound that modulates ceramide synthesis.

21. A compound identified by the method of claim 20.
